(19) United States
(12) Patent Application Publication
(10) Pub. No.: US 2015/0367020 A1
(43) Pub. Date: Dec. 24, 2015

(54) Methods of treating amniotic membranes using supercritical fluids and compositions and apparatuses prepared therefrom

(71) Applicant: Dorotea, LLC, San Antonio, TX (US)

(72) Inventors: Arnold L. ANDREWS, San Antonio, TX (US); William N. BORDANO, San Antonio, TX (US)

(21) Appl. No.: 14/737,217

(22) Filed: Jun. 11, 2015

Related U.S. Application Data
(62) Division of application No. 13/815,827, filed on Mar. 15, 2013.
(60) Provisional application No. 61/695,907, filed on Aug. 31, 2012.

(51) Int. Cl. A61L 15/40; A61L 27/54; A61L 27/38; A61L 15/44; A61L 27/36

(52) U.S. Cl. CPC A61L 15/40; A61L 15/44; A61L 27/3604; A61L 27/3834; A61L 27/54

(57) ABSTRACT

A method of sterilizing compositions prepared from amniotic membrane tissues may include harvesting placental tissue, separation of amniotic membrane tissue, and treatment of the amniotic membrane tissue with a supercritical fluid such as carbon dioxide. Treatment with supercritical fluid may subject the amniotic membrane tissue to conditions sufficient to sterilize the tissue yet maintain at least some biological function of the sterilized composition. The tissue preparations and compositions having a sterility assurance level of at least 10⁻⁶ described herein may be used as tissue grafts, wound dressings, cell culture substrates, or other substrates for use in tissue engineering.

### CROSS REFERENCE TO RELATED APPLICATIONS

The present application is a divisional of U.S. application Ser. No. 13/815,827, filed Mar. 15, 2013 to which priority is claimed. U.S. Ser. No. 13/815,827 is a utility application which claims priority to U.S. Provisional Application Ser. No. 61/695,907 filed Aug. 31, 2012.

### GOVERNMENT LICENSE RIGHTS

This invention was made with government support under a Cooperative Research and Development Agreement between Dorotea, LLC and U.S. Army Institute of Surgical Research dated 22 Aug. 2011. The government has certain rights in the invention.

### FIELD

The present application relates to methods of sterilization of biological tissues including amniotic membranes as well as to methods for loading such tissues with bioactive compounds. The application further relates to compositions prepared from amniotic membranes and the use of those compositions in apparatus such as wound dressings and/or surgical grafts.

### BACKGROUND

A number of biologically and medically relevant processes involve growth and/or migration of native cells or tissue into a surrounding region. For example, the migration of native cells into a region that has been subjected to traumatic injury may be an important step in the process of wound healing, and substrates that support such migration have been used to facilitate wound repair. More generally, various applications within the field of tissue engineering may depend upon the providing of a suitable support matrix upon which cells and tissues may integrate. For example, when allograft tissues are used in place of autografts the graft matrix or scaffold should provide a suitable environment for growth and/or differentiation of infiltrating cells and tissues. The amniotic membrane, the inner most portion of the placenta, includes a rich extracellular matrix, provides an abundance of biologically active proteins and other components to support tissue integration, and may be ideally suited for preparing compositions that are useful in tissue engineering. Such compositions have, for example, been used as biological dressings in numerous clinical wound healing, applications including the management′ of full and partial thickness burns, skin graft donor sites, and chronic leg ulcers.

The therapeutic effectiveness of amniotic membranes in such applications may be related to any of various characteristics of material derived from amniotic membranes such as, for example, the ability to stimulate the formation of granulation tissue, promote re-epithelialization of a wounded area, and do so with reduced scarring. In addition, such materials may possess inherent, anti-inflammatory and anti-immunogenic properties, which may diminish the risk of some complications that may occur during wound recovery. The aforementioned properties and characteristics may be related to a number of cellular and/or extracellular biological components of the amniotic membrane, including, for example, extracellular matrix proteins, cytokines, growth factors, and signaling molecules. Ideally, methods for preparing compositions that include amniotic membranes would preserve the biological activity of at least some of those components and, more importantly, maintain associated beneficial properties of those components in the wound healing process. For amniotic membranes which are both thin and fragile such preparations are extremely challenging.

In general, when a composition prepared from an amniotic membrane is used as a wound dressing, or graft, the composition may be subjected to a sterilization protocol. Ideally, a sterilization protocol would completely inactivate and/or remove any infectious agents that may be present, such as, e.g., viruses, bacteria, mycobacteria, mycoplasma, and fungi. Exemplary sterilization methods include the application of steam or dry heat, treatment with chemicals (such as, for example, ethylene oxide, and formaldehyde), use of ionizing and non-ionizing, sources of radiation, and combinations of those methods. Some sterilization techniques may, at least for some possible infectious agents, achieve a desired level of inactivation for example, a sterility level of about 106 or greater). However, methods for obtaining broad protection from the plurality of possible infectious agents that may be present in a given tissue sample are difficult to achieve. Moreover, adjusting method conditions to achieve broad protection from possible infectious agents may generally result in loss of at least some biological activity, including activity that may be beneficial in the wound healing process. Physical techniques like gamma irradiation and steam and heat sterilization cause significant structural damage through irreversible degradation of extracellular matrix proteins, and while valuable for sterilization of some materials physical techniques may be too severe for sterilization of thin membranous structures like the amniotic membrane. Some researchers have, for example, evaluated the chemical structure of steam and dry heat sterilized amniotic membrane grafts using infrared spectroscopy and found considerable differences in the infrared spectra of the sterilized group as compared to control samples See Rita Singh, Sumita Purohit, & M. P. Chacharkar, Effect of High Doses of Gamma Radiation on the Functional Characteristics of Amniotic Membrane, 76(6) Radiat. hys. Chern. 1026, 1026-1030 (2007). Other researchers have shown that sterilization with gamma irradiation results in destruction of the amniotic epithelium as well as dissolution of the compact and fibroblast connective layers. Consistent with such findings, it is probable that significant loss of desirable biological activity was initiated by sterilization of tissue using those methods. See F. von Versen-Hoynck, C. Syring, S. Bachmann, & D. E. Moller, The influence of different preservation and sterilization steps on the histological properties of amnion allografts—light and scanning electron microscopic studies, 5(1) Cell Tissue Bank, 45, 45-56 (2004). The detrimental effects that occur as a result of sterilization treatment have the potential to alter or impair the desired function of the tissue post implantation.

To date, a method for treatment of amniotic membranes that achieves both inactivation and/or removal of the possible infectious agents that may be present in a sample of amniotic membrane tissue and which maintains beneficial biological properties of the tissue has not been realized.

### SUMMARY

A method of preparing a sterilized composition may include harvesting tissue from at least one placenta thereby producing a harvested tissue, wherein the harvested tissue includes one or more microbes; isolating amniotic membrane tissue from the harvested tissue; and treating the amniotic membrane tissue with a supercritical fluid at a temperature and pressure for a period of time sufficient to sterilize the amniotic membrane tissue to a specified assurance level of sterilization with respect to the one or more microbes, thereby producing the sterilized composition from the amniotic membrane tissue.

A method of producing a sterilized composition may include harvesting tissue from at least one placenta, wherein the harvested tissue includes bacteria to be inactivated, isolating amniotic membrane tissue from the harvested tissue, freezing the amniotic membrane tissue, thawing the amniotic membrane tissue, and contacting the amniotic membrane tissue with a supercritical fluid at a temperature and pressure for a period, of time sufficient to achieve an assurance level of inactivation of the bacteria of at least 6 log orders.

A method of producing a sterilized composition may include harvesting tissue from at least one placenta, wherein the harvested tissue includes bacteria to be inactivated, isolating amniotic membrane tissue from the harvested tissue, subjecting the amniotic membrane tissue to a treatment wherein membranous structures of the tissue may be damaged and/or ruptured and contacting the amniotic membrane tissue with a supercritical fluid at a temperature and pressure in the presence of an oxidant, such as peracetic acid, for a period of time sufficient to achieve an assurance level of inactivation of the bacteria of at least 6 log orders.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of a system for treating amniotic membrane tissue with a supercritical fluid.

FIG. 2 is a flowchart depicting a method of sterilizing a composition.

FIG. 3 is a flowchart depicting some steps that may be present in a part of a method of sterilizing a composition.

FIG. 4 is a flowchart depicting a method of sterilizing a composition and impregnation of the composition with one or more bioactive agents.

FIGS. SA-F are light micrographs of native (Fig. SA, SC, and SE) and supercritical carbon dioxide-treated amniotic membrane samples (Fig. SB, SD, and SF).

FIGS. 6A-6C are a set of light micrographs of supercritical carbon dioxide-treated amniotic membrane samples exposed to different treatment times.

FIGS. 7A-7B are representative scanning electron microscopy images of the epithelium of native (FIG. 7A) and supercritical carbon dioxide-treated amniotic membrane tissue (FIG. 7B). FIGS. 7C-7D are representative fluorescence images of epithelium of native (FIG. 7C) and supercritical carbon-dioxide treated amniotic membrane tissue (FIG. 7D).

FIG. SA is a set of thermograms of denatured, native, and supercritical carbon dioxide-treated amniotic membrane tissue. FIG. 8B is a set of infrared spectral graphs for both native amniotic membrane tissue and supercritical carbon dioxide-treated amniotic membrane tissue.

FIGS. 9A-9D are graphs of results of biochemical assays for hydroxyproline (FIG. 9A), collagen IV (FIG. 9B), sGAG (FIG. 9C), and elastin (FIG. 9D) content of native and supercritical carbon dioxide-treated amniotic membrane tissues determined using colorimetric methods and/or enzyme-linked immunosorbent assays.

FIG. 10A is a representative fluorescent micrograph of human adipose-derived stem cells (hASC) seeded on supercritical carbon dioxide-treated amniotic membrane tissue after 24 h of culture. FIG. 10B is a chart showing hASC proliferation monitored with an MTT assay.

FIG. 11 is a graph of percentage wound closure of full thickness excision wounds treated with either saline, amniotic membrane tissue wound dressing, or hASCs-amniotic membrane wound dressing.

FIG. 12 is a set of light micrographs showing full thickness excision wounds treated with saline, amniotic membrane tissue wound dressing, or hASCs-anmiotic membrane tissue wound dressing monitored over a period of 16 days.

FIGS. 13A-13B are a set of high resolution micrographs detailing the epidermis of normal rat skin (FIG. 13A) and the epidermis of a wound treated with the amniotic membrane tissue wound dressing after 12 days of healing (FIG. 13B).

FIG. 14 is a high magnification image of an MTS stained tissue section after 12 days of healing.

### DETAILED DESCRIPTION

The following terms as used herein should be understood to have the indicated meanings.

When an item is introduced by “a” or “an,” it should be. understood to mean one or more of that item.

“Comprises” means includes but is not limited to.

“Comprising” means including but not limited to.

“Having” means including but not limited to.

“Sterilize” means the removal or inactivation of one or more microbial organisms or agents (herein referred to as “microbes”), such as yeast, fungi, bacteria, viruses, spores, or the like, from or in an article, either completely or to a specified degree.

The term “supercritical fluid” as used herein means a fluid at or above the critical temperature and critical pressure of the fluid.

The term “tissue engineering” as used herein refers to the design, manufacture, and use of cells and/or tissues to improve, repair, or replace a tissue that has been damaged or lost.

This disclosure is generally directed to methods of sterilizing compositions prepared from amniotic membrane tissue and the use of compositions thereof in tissue engineering. This disclosure is further directed to sterilized compositions prepared from amniotic membrane (AM) tissue. Various uses of the sterilized compositions described herein may include, by way of non-limiting example, use as any of various wound dressings, use as delivery vehicles for bioactive/therapeutic compounds, and use as substrates for cell culture, including, but not limited to, stem cell expansion. Amniotic membrane tissue from human or other mammalian species may be used as described herein.

The methods described herein may include obtaining amniotic membrane tissue and treating the tissue with a supercritical fluid. Treatment with supercritical fluid may involve subjecting the tissue to conditions sufficient to inactivate and/or remove infectious materials that may be present, load a bioactive or therapeutic compound, modulate therapeutic properties of the resultant sterilized composition (such as immunogenicity), or achieve combinations of the aforementioned results. Moreover, in some embodiments, supercritical fluid treatment may involve the selection of conditions that minimize damage to tissue components, such as extracellular matrix proteins, that may provide desired•biological or therapeutic properties.

In some embodiments, amniotic membrane tissue samples (or a portion of such samples) before and after treatment with supercritical fluid may be measured, such as using spectrographic methods (such as infrared absorption), differential scanning calorimetry or other techniques. Spectral or other measurements thereof may be analyzed and used to qualify the samples. In addition, therapeutic outcomes associated with the use of compositions prepared from amniotic membrane tissue and treated with a supercritical fluid as described herein may be compared to therapeutic outcomes for other wound dressings including amniotic membrane tissue sterilized using other sterilization methods. In some embodiments, spectral characteristics or thermal characteristics of the sterilized compositions may be used to qualify and/or reject a given tissue for use as a composition in a wound dressing.

An amniotic membrane tissue sample may, in some embodiments, be qualified for use as a clinical wound dressing if a portion of the tissue exposed to sterilization treatment (when compared with a control sample) is characterized as acceptable based on one or more characteristics' of one or more thermal transitions measured with calorimetry. For example, as further described in relation to the calorimetric data of Example 4, a first thermal transition (between about 100° C. and 150° C.) and a second thermal transition (between about 200° C.) and 240° C.) may be used to characterize amniotic membrane tissue samples. Either or both of those transitions may, for example, be used to qualify a tissue as having maintained suitable biophysical properties during, processing and then used•in clinical applications. A thermal transition may, for example, be characterized by a magnitude of heat flow or by a temperature at which heat flow occurs, such as, by way of nonlimiting example, thermogram peak intensity, peak height, transition temperature, transition temperature width, or any combinations thereof hi some embodiments, characteristics of a first thermal transition (which is characteristic of the loss of bound water and may be a highly sensitive probe for structural and/or chemical changes in an AM tissue sample) may be used to qualify tissue. For example, in some embodiments, the transition temperature of pre- and post-sterilization treated tissue samples may differ by less than 2° C. less than 4° C., less than 5° C. or by some other value to be qualified for clinical applications.

A supercritical fluid may possess a combination of properties that are typically found in gases and other properties typically found in liquids. For example, a supercritical fluid may possess the ability to penetrate some solids, such as porous solids (a property typical of gases), and a supercritical fluid may also have the ability to dissolve a wide range of solutes (a property typical of liquids). Carbon dioxide, a material that is non-toxic (when residually present), readily available, non-flammable, dissolves a wide range of solutes, and possesses a relatively low critical temperature, may be a principal supercritical fluid used in methods described herein. The critical temperature of carbon dioxide is about 31. ° C., which is significantly lower than a number of other materials that have been used as supercritical fluids, Therefore, supercritical carbon dioxide (SCC02) may be used in methods of treating components that may be thermally labile and where preservation of some level of biological activity may be desired.

In some embodiments, supercritical carbon dioxide may be the sole solvent in a treatment system. In other embodiments, supercritical carbon dioxide may be used along with one or more other solvents. For example, in some embodiments, a solvent may be used to modify the solubility, or rate of dissolution, of a bioactive or therapeutic reagent that may be impregnated within a composition prepared from amniotic membrane tissue. A solvent may, for example, be added along with supercritical carbon dioxide by pumping the solvent into a treatment vessel during carbon dioxide filling, added directly to the composition for treatment (such as by soaking amniotic tissue in one or more liquid phase solvents), added to the treatment system in a combination of those ways, or added in some other manner.

The sterilized compositions described herein may include at least a portion of one or more amniotic membranes. The amniotic membrane is the innermost portion of the placenta, includes a thin layer of epithelial cells, a thicker basement membrane, and several layers of stromal tissue. As noted above, the amniotic membrane includes a rich extracellular matrix; provides an abundance of biologically active components, such as proteins, and may be•ideally suited for preparing compositions for use in tissue engineering. In some embodiments, it may be desirable to limit the application pressure, temperature, and/or time of exposure of a tissue to a supercritical fluid in order to maintain some level of biological activity. A preparative treatment (before application of supercritical fluid) may be used to devitalize a tissue sample and may, in some embodiments, also prepare or sensitize a composition for sterilization, in some embodiments, a treatment (or treatments) may prepare or sensitize a tissue for sterilization using supercritical fluid and an oxidant, such as peracetic acid. Sensitization of a prepared tissue may be characterized by achieving with a certain set of conditions (such as supercritical fluid temperature, time, pressure or concentration of peracetic acid) a given level of sterility assurance that is greater than would otherwise be found without preparation. Sensitization of a prepared tissue may also be characterized by the achieving of a certain level of sterility assurance with a set of conditions (such as supercritical fluid temperature, time, pressure or concentration of peracetic acid) that is less severe, for example, lower supercritical fluid temperature, time, pressure or concentration of peracetic acid, than otherwise may be achieved without sensitization or preparation.

The combination of tissue preparation and supercritical fluid treatment may achieve a desired level of sterilization and maintain a desired level of biological and/or therapeutic activity for some or all components of the tissue. For example, in some embodiments, subjecting amniotic membrane tissue to one or more freeze-thaw cycles, supercritical fluid treatment (in the presence of peracetic acid), or combinations thereof may facilitate a desired level of sterilization yet maintain beneficial wound healing characteristics of the tissue.

Preparative treatment or treatments may damage a tissue's cell membranes and/or cause lysis of at least some of the tissue's cells. For example, in some embodiments, amniotic membrane tissue may be subjected to at least one freeze-thaw cycle that may weaken and/or destroy membranous components of cells in the tissue or damage a type of cell in the tissue, such as epithelial cells. A tissue may be frozen by refrigeration of the tissue, such as by subjecting the tissue to a temperature of about −80° C. or some other suitably low temperature. In some embodiments, a tissue may be subjected to a temperature of less than about −20° C. In some embodiments, preparative treatment of the tissue may include freezing the tissue without use of a cryoprotectant, such as glycerol. A tissue may be thawed by subjecting a tissue to ambient temperature conditions or to some other•suitably warm condition. In some embodiments, the rate of temperature change may be controlled by subjecting tissue samples to freezing and/or thawing in a step-wise or gradient manner. In some embodiments, addition of one or more freeze-thaw cycles may serve to enhance the effectiveness by which supercritical fluid facilitates penetration of an oxidizing agent, such as peracetic acid, within a tissue. Increasing the rate of oxidant penetration (or uniformity of penetration within a tissue) may facilitate inactivation and/or removal of infectious material using less severe supercritical fluid conditions and/or lowered amounts of oxidant to achieve a given level of sterility.

During a freeze-thaw cycle of amniotic membrane tissue, ice crystals may accumulate within cells thereof, the cells may swell, and the outer cell membrane or other membranous structures of a given cell may become weakened or may rupture. Following a freeze-thaw cycle, the composition may be devitalized and at least some possible contaminants may be inactivated and/or sensitized to further treatment, such as treatment with a supercritical fluid, which may or may not be in the presence of an oxidizing agent. A prepared tissue may, in some embodiments, exhibit an increased porosity and lowered density. In some embodiments, the rate of freezing and/or the rate of thawing of a composition may be controlled, such as by bathing the composition in a liquid solution, such as saline, while thawing. A solution may serve as a thermal sink and may modify the rate of heat flow and temperature change. In some embodiments, a rate of heat transfer, i.e., transfer between a composition and the environment, may be controlled such that the rate of temperature change may be about 1° C. per minute or another suitable rate.

Prior to a given freeze-thaw cycle and/or treatment with supercritical fluid, an amniotic membrane tissue may, in some embodiments, be washed, such as with a physiological buffer solution. In some embodiments, a wash buffer may be′ supplemented with an antibiotic and/or antimycotic solution. Alternatively, in some embodiments, no antibiotic or antimycotic agent may be added (at least prior to treatment). Following washing, the amniotic membrane may, in some embodiments, be placed epithelial side up on nitrocellulose paper, and may be stored until further processing. For example, the membrane may be stored at about 4° C. or some other suitable temperature until further processing.

In some embodiments, preparation of amniotic membrane tissue may alternatively or additionally involve subjecting the tissue to a treatment that removes native DNA from the tissues cells. Removal of DNA may, in some embodiments, be achieved by subjecting the tissue to an endonuclease enzyme, i.e., an enzyme that may cleave phosphodiester bonds within DNA. While removal of DNA may be used in combination with supercritical fluid treatment to sterilize amniotic membrane tissues (and it may be desirable to do so), some embodiments described herein may not include enzymatic treatment to remove native DNA. For example, as described in relation to Examples 1-6, treatment of amniotic membrane tissues using a peracetic acid containing solution diluted to about 0.01% vol/vol and with an unexpectedly brief exposure time of supercritical carbon dioxide (for example, only about 10 minutes) may achieve a sterility level of assurance of 10⁻⁶ (that is, a 106 reduction in bacterial load) without including a dedicated step for enzymatic remove native DNA. Moreover, conditions in those Examples 1-6 were found to achieve a 10⁶ reduction in load of spore-forming bacteria (which are typically difficult to remove) and do so without significantly altering tissue architecture, the amounts of pertinent extracellular matrix proteins (type IV collagen, glycosaminoglycans, elastin) present in the tissue, or the biophysical properties of the tissue. For some applications, savings of cost and time realized through the use of sterilization protocols without specific enzymatic digestion of native DNA may be significant.

As noted above, freeze-thawing and/or removal of DNA may devitalize amniotic membrane tissue and/or prepare the tissue for sterilization with a supercritical fluid, such as supercritical carbon dioxide. Supercritical carbon dioxide may be provided as a substantially purified reagent, such as carbon dioxide of greater than about 99% purity. Supercritical carbon dioxide may be used at or above the critical temperature and pressure of the fluid (31° C., 1070 psi). In some embodiments, the temperature of supercritical carbon dioxide may be between about 32° C. and about 38° C. and the pressure of supercritical carbon dioxide may be between about 1350 psi and about 1500 psi. In some embodiments, the temperature of supercritical carbon dioxide may not exceed 50° C., 45° C., or 38° C. In some embodiments, the pressure of a supercritical carbon dioxide fluid may not exceed 1800 psi, 1600 psi, or 1500 psi. Selection of a given temperature, pressure, and/or treatment time may minimize the risk of loss of activity of extracellular matrix proteins or other tissue components that may be present. Moreover, if the severity of conditions is too extreme, structural integrity of thin and fragile amniotic membranes may be compromised. For various applications in wound healing, loss of structural integrity may compromise the performance of the membrane. In some embodiments, a composition may be in contact with supercritical carbon dioxide for about 10 minutes to about 60 minutes, or about 20 minutes to about 30 minutes, or about 10 minutes to about 20 minutes. In some embodiments, a combination of supercritical carbon dioxide and peracetic acid sterilant may be used and a composition may be in contact with supercritical carbon dioxide for no more than 30 minutes, such as between about 8 minutes and about 15 minutes.

In some embodiments, a supercritical fluid, such as carbon dioxide, may contact amniotic membrane tissue during sterilization treatment and in the presence of peracetic acid. Peracetic acid is an oxidizing agent that exhibits antimicrobial activity to a wide variety of microbes. A diluted peracetic acid solution may, in some embodiments, be added directly to the tissue, such as a devitalized tissue sample, and the tissue may then be treated with supercritical fluid and sterilized.

In some embodiments, peracetic acid may come in contact with amniotic membrane tissue only when carried to the tissue as a solute dissolved in supercritical fluid. For example, peracetic acid may be soaked within a membrane, porous support material, or other suitable support material that is present within a chamber used for supercritical fluid treatment but physically separated from the tissue for treatment. A suitable support material may contain the peracetic acid and present the solution for solvation by supercritical carbon dioxide. For example, peracetic acid may be soaked into a pad that may be located at the bottom of a supercritical fluid chamber, or some other location in the chamber, and the supercritical fluid may be introduced within the chamber, flow through the pad, solvate the peracetic acid, and carry the acid to the tissue composition for sterilization. Therefore, the peracetic acid may contact tissue intended for sterilization only after significant dilution and while in the presence of carbon dioxide solvent. In some embodiments, providing peracetic acid in this manner may act to minimize a dose of peracetic acid that may come in contact with amniotic membrane tissue, yet still provide effective tissue sterilization while maintaining components of tissue architecture and/or biochemical properties of the native amniotic tissue. In some embodiments, about 0 milliliters to about 10 milliliters, about 0 milliliters to about 4 milliliters, or about 2 milliliters of an about 35% to about 40% peracetic acid solution may be soaked into an absorbent pad and following solvation with supercritical carbon dioxide the peracetic acid may permeate within about a 22 liter chamber. Of course, other dimensions and other amounts of peracetic acid may be used, such as from about 0 ml of diluted peracetic acid per liter of chamber to about 0.5 ml of diluted peracetic acid per liter of chamber—for a diluted peracetic acid of about 35% to about 40% concentration by weight. For example, in some embodiments, about 39% purity peracetic acid may be used, which is available from Sigma-Aldrich (St. Louis, Mo.) and available under the Sigma-Aldrich catalog number 77240. The peracetic acid, from that source, may include less than about 6% hydrogen peroxide and may include about 45% acetic acid. In some embodiments, about 0 grams to about 4.5 grams of peracetic acid may be added to an about 22 liter chamber. In some embodiments, about 0 grams to about 0.2 grams of peracetic acid may be added per liter of chamber.

An amount of peracetic acid may also be conveniently expressed as a percentage based on the volume of a peracetic acid containing solution used and the volume of the chamber in which the solution is diluted. For example, 2 ml of a peracetic acid solution (which as described above may be provided from a solution of about 35% to about 40% concentration by weight) may be diluted in an about 20 liter container thereby providing the peracetic acid containing solution at about 0.01% vol/vol. In some embodiments, a peracetic acid containing solution may be diluted to between about 0.0025% vol/vol to about 0.015% vol/vol.

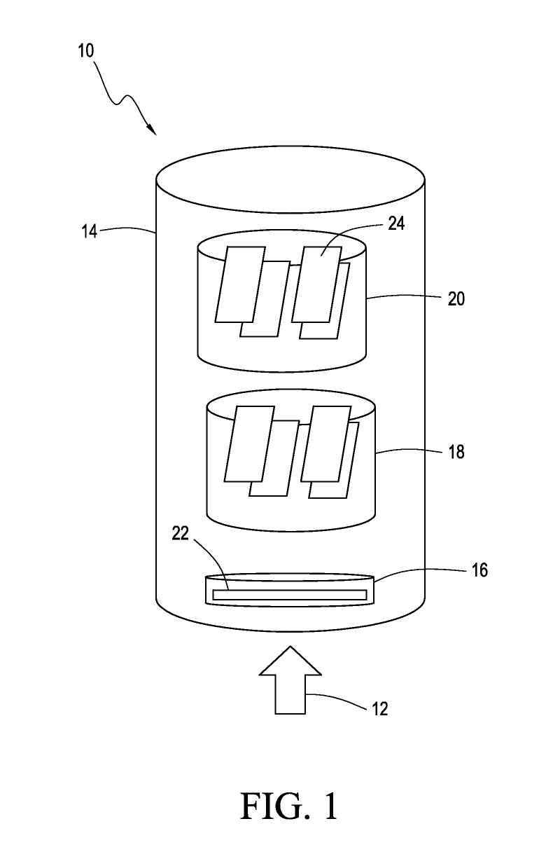

FIG. 1 illustrates a system 10 for supercritical fluid treatment of amniotic membrane tissue. As shown in FIG. 1, a flow of supercritical fluid 12 may be introduced into a process chamber 14, such as at the bottom of the process chamber 14. The process chamber 14 may include any number of containers (16, 18, and 20) for holding material to be treated or other processing reagents. For example, the process chamber 14 may include a bottom container 16, a center container 18, and a top container 20. The bottom container 16 may contain a porous material 22, which, as described above, may be soaked with peracetic acid. The center container and top container 20 may serve to hold a number of samples of amniotic membrane tissue 24.

Supercritical carbon dioxide may be ideally suited to penetrate amniotic membrane tissue, solubilize, and facilitate transport of biological contaminants and cellular debris from exposed tissue. If the tissue is porous, low in density, and/or thin, penetration of supercritical fluid within the tissue may be enhanced. In some embodiments, supercritical fluid may be used to facilitate interpenetration of the oxidant peracetic acid within the amniotic membrane tissue. Peracetic acid may oxidize any of various biological components of contaminant infectious organisms, such as membranous compounds and other components. Oxidation may facilitate deactivation and/or lysis of microbial elements, and resultant cellular and/or tissue fragments may be solubilized and carried by the supercritical fluid away from the sterilized tissue.

Methods described hereitl may, in some embodiments, achieve a sterilization assurance level of at least 10⁻⁶; i.e., a 6 log reduction in colony forming units per milliliter (CFU/ml). Other suitable sterilization levels may also be achieved, depending on the microbial agents of interest and the needs of a particular application. Sterilization assurance levels may be achieved for any of various infectious agents, including, by way of nonlimiting example, viruses, retroviruses, yeasts, fungi,) bacteria (including gram positive bacteria, gram negative bacteria, aerobic bacteria, anaerobic bacteria, and spore-forming bacteria), mycobacteria, mycoplasma, and combinations thereof. Examples of viruses include Murine Leukemia virus, Hepatitis A virus, Hepatitis B virus, Hepatitis C virus, and Human immunodeficiency virus. Examples of fungi include Candida albicans, Pneumocystis jirovecii, and Cryptococcus neoforman. Examples of bacteria include Escherichia Coli, Staphylococcus aureus, Treponema pallidum, Pseudomonas aeruginosa, Streptococcus pyogenes, Clostridium sporogenes, Staphylococcus epidermidis, and Bacillus atrophaeus.

In some embodiments, a sterilization level of assurance may be verified through the analysis of a suitable number of test samples, such as amniotic membranes, in which a target infectious agent may be added or may be known to be present. In some embodiments, one or more infectious agents may be added to one or more samples and verification may be made that each of the infectious agents has been removed or inactivated. Verification of a level of sterilization assurance may, for example, include addition of a target infectious agent to a suitable number of test samples, such as by addition of infectious agent to a concentration near or above about 106 colony forming units per milliliter. The test samples may be subjected to sterilization protocols described herein and analyzed for the presence of the target infectious agent Any suitable analytical technique sensitive to the presence of the target infectious agent, such as a suitable microscopic analysis, staining, labeling, or some other protocols, may be used. In some embodiments, the test samples (following a sterilization protocol) may be subjected to cell culture conditions known to be suitable for growth of the target infectious agent, and then suitably analyzed for the presence of the infectious agent. Using sterilization with supercritical fluid treatment, as described herein, a level of sterility of greater than 106 may, in some embodiments, be obtained, even for bacterial species known to be spore-forming, which typically are challenging to remove and/or inactivate.

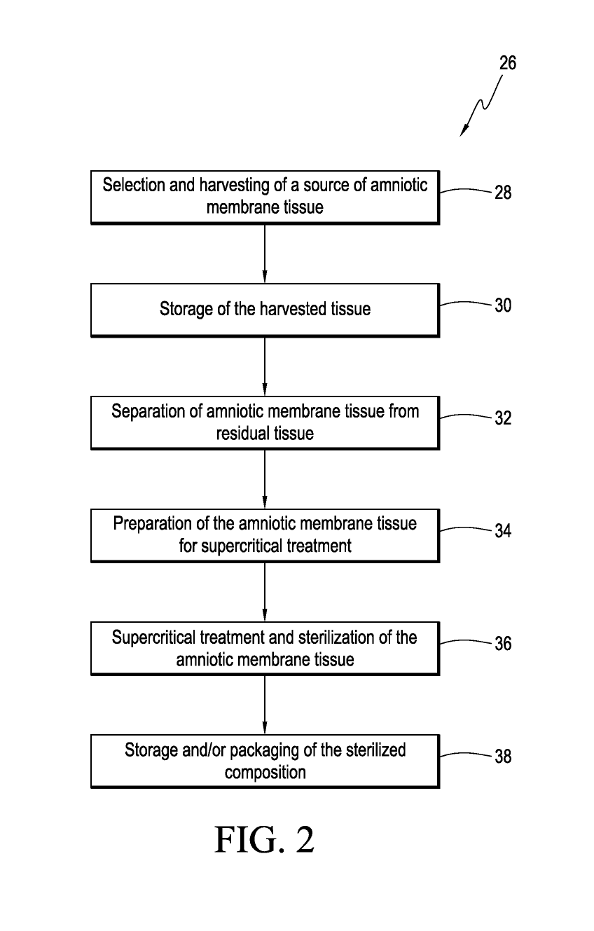

FIG. 2 depicts an exemplary embodiment of a method 26 of preparing a sterilized composition from amniotic membrane tissue. In a step 28, a source of amniotic membrane tissue may be selected and harvested for use. In some embodiments, placentas from mothers undergoing natural birth or elective caesarian section delivery of term babies may be harvested. Donors may, in some embodiments, be screened for infectious disease including, by way of non-limiting example, HIV, hepatitis A, hepatitis B, syphilis, or other infectious disease using available serological methodologies. Harvesting tissue may include cutting the placenta or a portion of the placenta from surrounding connective tissue. In some embodiments, the harvested tissue may be transported to a processing facility for further dissection and processing. The transport of tissue may involve sealing the harvested tissue in a sterile container on wet ice and shipping the tissue.

In a step 30, harvested tissue may be stored for at least some period of time prior to further use. For example, upon arrival at a processing facility, placental tissue may be stored under hypothermic conditions. Placental tissue may, for example, be stored at about 4° C. for up to about 72 hours prior to being aseptically processed in a clean room. In some embodiments, storage may comprise holding harvested tissue at about 2° C. to about 6° C. for a maximum acceptable period of time, such as no more than about 96 hours, no more than about hours, or no more than about 48 hours, for example. In other embodiments, harvested material may be processed as rapidly as possible, such as substantially upon arrival at the processing facility and without substantial storage.

In a step 32, amniotic membrane tissue may be separated from residual tissue of the harvested material. Separation of the amniotic membrane tissue from residual tissue may include rinsing the harvested tissue with a wash solution prior to or during peeling and/or cutting of amniotic membrane tissue from the chorion and/or other tissues, such as deciduous layers, using blunt dissection or other suitable methods. In some embodiments, wash fluid may be a saline solution that may be supplemented with an antibiotic agent, antimycotic agent, pH buffer, other ingredient or any combination thereof. Blood clots and/or other residual material that may remain following dissection may be further rinsed, such as in saline, and the rinsed amniotic membrane material may be placed on a support medium, such as sheets of nitrocellulose paper or some other suitable medium, such as a porous support matrix. The removal of residual tissue may, in some embodiments, facilitate orientation of the amniotic membrane in a desired manner, such as, for example, as a substantially planar sheet. The amniotic membrane tissue may, in some embodiments, be placed with the epithelial surface facing upwards, i.e., away from the support medium, and generally laid as a sheet. An amniotic membrane may be about 20 to about micrometers in thickness. The membrane may, in some embodiments, be placed with the stromal side of the membrane attached to the support medium, leaving the epithelial side facing upwards. In some embodiments, the substantial planarity of the tissue may be validated by visual inspection and/or some other technique; Configuring the tissue in a substantially planar configuration may help facilitate homogenous, isotropic penetration of supercritical fluid solvent and/or peracetic acid (as discussed in step 36), thereby achieving effective sterilization of all portions of the membrane without excessive contact of some portions of the tissue. Orienting the epithelial surface upwards may help preserve stromal tissue layers during sterilization.

In a step 34, the amniotic membrane tissue may be prepared for treatment with supercritical fluid. In some embodiments, preparation may involve storage of tissue at a suitable temperature, such as about 4° C. or another suitable temperature, until further processing. The preparation of tissue may, in some embodiments, be performed without the use of detergents, the use of which, may, at some detergent concentrations, lessen the therapeutic effectiveness of the final sterilized composition. The preparation step 34 may also, in some embodiments, involve one or more treatments wherein the tissue may be devitalized, including, for example, subjecting the tissue to a freeze-thaw stress, other treatments or combinations thereof. Preparative step 34 may, in some embodiments, act to weaken and/or lyse membranous elements of the tissue cell structure. Freezing the amniotic membrane tissue may involve reducing the temperature around the amniotic membrane tissue to a temperature of less than about −20° C., including, for example, a temperature of about −80° C., such as by placing the tissue in a refrigeration unit. The frozen amniotic membrane tissue may be stored until further use or in some embodiments for a period of at least about 24 hours. The frozen amniotic membrane tissue may be thawed by removing the amniotic membrane tissue from the refrigeration unit and placing the tissue in a suitable temperature controlled environment. In some embodiments, the tissue may be removed from the refrigeration unit and allowed to thaw while at room temperature. During freeze-thawing, ice crystals may form, expand, and promote disruption of the amniotic membrane cellular structure. At least some of the cellular constituents that may be present in the amniotic membrane may become non-viable; however, the tissue matrix may remain substantially intact. In some embodiments, following or prior to freeze-thawing, the amniotic membrane may be sealed in porous, sterile packaging. For example, the amniotic membrane may be sealed in porous, medical grade Tyvek™ packaging available from E.I. duPont de Nemours and Company (Wilmington, Del.).

In a step 36, the amniotic membrane tissue or packaged amniotic membrane tissue may be loaded into a process chamber, such as a Nova 2200 Sterilization System available from NovaSterilis, Inc. (Lansing, N.Y.), configured for use with conditions associated with supercritical fluids and treated with supercritical fluid such as supercritical carbon dioxide. In some embodiments, the packaged amniotic membrane tissue may be held within a container, such as a mesh basket, and mounted within the chamber (see FIG. 1): The container, packaging (such as Tyvek™ packaging), and support medium upon which tissue is laid or attached (such as nitrocellulose paper), may serve to support the packaged amniotic membrane tissue and may be configured to allow fluid to contact the packaged amniotic membrane from either the epithelial or opposing side, such as without substantial obstruction of fluid flow.

The supercritical fluid may be loaded into the chamber, such as through the chamber bottom, and may, in some embodiments, flow through a pad or other suitably porous material soaked with an oxidizing agent, such as peracetic acid. The supercritical fluid may solvate and•facilitate transport of peracetic acid through the chamber and into contact with the amniotic membrane tissue. Moreover, the supercritical fluid may perfuse peracetic acid through the tissue structure facilitating contact and effective deactivation of contaminant material that may be present in the amniotic membrane tissue.

Supercritical carbon dioxide may be used at or above the critical temperature and pressure of the fluid (31° C., 1070 psi). For example, in some embodiments, the temperature of supercritical carbon dioxide may be between about 32° C. and about 38° C. and the pressure of supercritical carbon dioxide may be between about 1350 psi and about 1500 psi. In some embodiments, the method may include an amount of carbon dioxide of about 800 grams of carbon dioxide per gram of tissue to about 900 grams of carbon dioxide per gram of tissue. In some embodiments, the composition may be in contact with supercritical carbon dioxide for about 10 minutes to about 60 minutes or other suitable time period.

In a step 38, the sterilized composition produced from supercritical fluid treatment step 36 may be stored and/or packaged. For example, upon completion of supercritical fluid treatment the composition may be stored frozen, such as at −80° C. or some other suitable temperature, or the composition may be lyophilized and stored under other temperatures, including room temperature or elevated temperatures (e.g., normal body temperature or above). In some embodiments, a portion of the sterilized tissue may also be qualified prior to use. For example, a portion of the tissue may be removed from other portions, subjected to one or more tests (for example, FTIR or calorimetry) and may then be characterized to be suitable for clinical use.

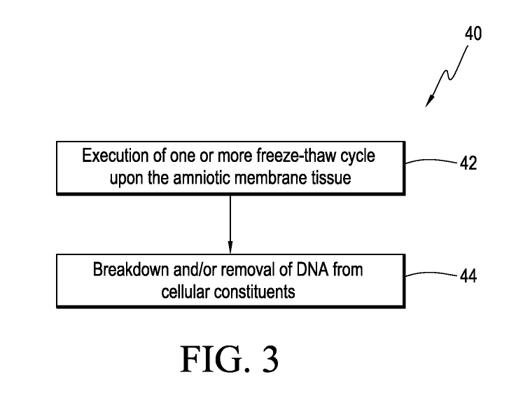

Method 26 may provide a sterilized composition that may be used in various applications in the field of tissue engineering, including, for example, use as a wound dressing, use as substrates in cell culture, and other uses, In some embodiments, the sterilized composition may be used in surgical repair and/or replacement of a damaged cornea in a patient. As noted above, the preparative step 34 may include one or more treatments wherein a tissue may be devitalized, including, for example, subjecting the tissue to one or more freeze-thaw cycles. In some embodiments, as depicted in FIG. 3, preparative process 40 may involve a process wherein DNA from cellular constituents that may be present may be broken down and/or removed. Some embodiments of a preparative process 40 wherein DNA is broken down and/or removed from cellular constituents in a tissue sample are depicted in FIG. 3.

As shown in FIG. 3, the preparative process 40 may include a step 42 involving one or more freeze-thaw cycles and a step 44 wherein DNA from cellular constituents in a tissue sample may be removed and/or broken down. In some embodiments, in step 44, amniotic membrane tissue may be bathed in a buffered solution including the endonuclease enzyme DNase 1, such as at a concentration of about 1,000 units per milliliter or other suitable concentrations. In some embodiments, endonuclease may be included at a concentration of about 100 units per milliliter to about 1000 units per milliliter. In some embodiments, the buffered solution may include about a 50 millimolar solution of the tris(hydroxymethyl)aminomethane, such as at a pH of about 7.5 or other suitable pH. Other additives, such as magnesium chloride at about 10 millimolar, or other suitable concentrations, buffer concentrations, enzyme concentrations, and pH values may be used as appropriate to optimize or maintain a desired level of endonuclease reaction efficiency. A tissue sample may, in some embodiments, be held at about 37° C. for about 3 hours or other suitable conditions may be used. For example, the rate of transport of endonuclease to any nuclear material that may be present in a tissue may depend upon whether or not the tissue has been previously subjected to one or more freeze-thaw cycles. In addition, in some embodiments, periodic agitation and/or sonication may be applied to the reaction buffer solution to enhance mixing.

In some embodiments, it may be advantageous to load an amniotic membrane tissue sample with one or more bioactive agents. As previously noted, supercritical carbon dioxide may be ideally suited for facilitating the penetration of an oxidant species, such as peracetic acid, within a tissue. More specifically, a supercritical fluid may possess various properties including, for example, low viscosity and low surface energy which may assist the fluid in accessing porous spaces within the tissue. A supercritical fluid may also be highly compressible, and the relative concentration of a dissolved solute, such as a bioactive agent, may be adjusted by modification of the pressure of the supercritical fluid. Furthermore, in some embodiments, the addition of a bioactive agent within a tissue may be adjusted by addition of an amount of cosolvent.

In addition to oxidant species, such as peracetic acid, bioactive agents that may be added to a tissue using supercritical fluid impregnation include, by way of nonlimiting example, antibiotics, therapeutic drugs (such as, anti-inflammatory and analgesic compounds), proteins, and growth factors. Examples of antibiotics that may be added include aminoglycosides, such as gentamicin, and glycopeptides, such as vancomycin.

In some embodiments, before supercritical fluid treatment, amniotic membrane tissue may be soaked in a solution including one or more bioactive agents intended for addition. In some embodiments, the solution of bioactive agent(s) may be in contact with the tissue for a period of time of between about 10 minutes to about 24 hours, or for some other desired period of time. A solution including the one or more bioactive agents may be an aqueous solution or other solvents may be used. In some embodiments, additional reagents may be added to the liquid solution, such as to improve the solubility of a bioactive agent in the desired solvent, stabilize a bioactive agent, or facilitate impregnation of a bioactive agent within the tissue sample. For example, in some embodiments, the solution of bioactive agent(s) may include chitosan microspheres or micelles. The addition of hitosan microspheres or micelles may, in some embodiments, provide a controlled release of a certain drug, antibiotic, or other bioactive agent.

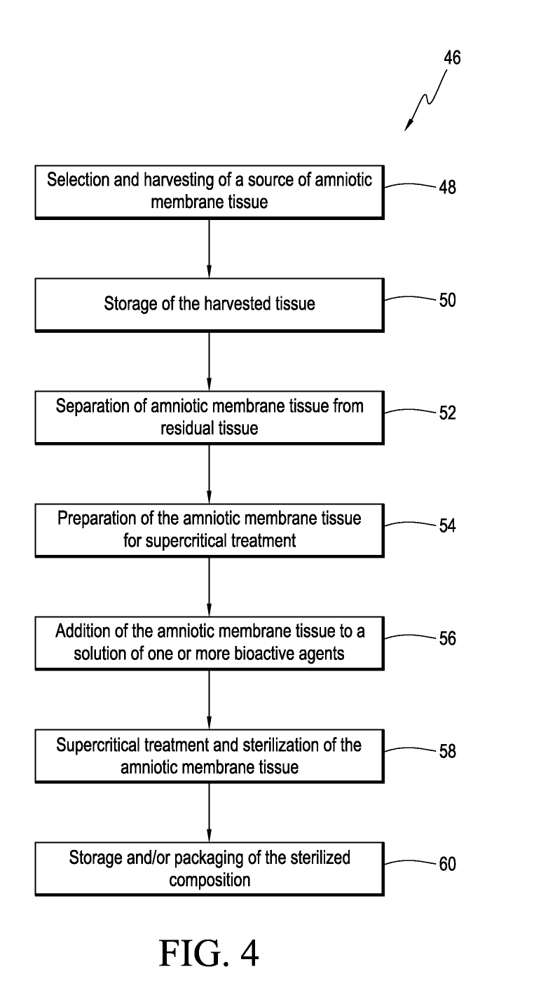

FIG. 4 depicts an exemplary embodiment of a method 46 of preparing a sterilized composition from amniotic membrane tissue and loading a bioactive agent in the tissue. In a step 48, a source of amniotic membrane tissue may be selected and harvested for use. In a step 50, if desired harvested tissue may be stored until further processing. In a step 52, amniotic membrane tissue may be separated from other harvested tissue. In a step 54, preparative steps may be executed. For example, as previously discussed, a sample of amniotic membrane tissue may be subjected to one or more freeze-thaw cycles.

In a step 56, a solution of one or more bioactive agents may be added to amniotic membrane tissue. In some embodiments, the amniotic membrane and associated support medium, such as nitrocellulose paper, may be soaked in a pan or other suitable container that includes the solution of one or more bioactive agents. Following a period of time in which the tissue is soaked, the tissue may be sealed in Tyvek™ packaging. In other embodiments, the tissue and solution of bioactive agents may be added to the Tyvek™ packaging, the packaging may be sealed, and the tissue and solution of bioactive agents may be contacted for a desired period of time, within the sealed Tyvek™ packaging.

In a step 58, packaged amniotic membrane tissue may be loaded into a process chamber configured for use with conditions associated with supercritical fluids and supercritical fluid added. As discussed previously, in some embodiments, an oxidant such as peracetic acid may also be added. In a step 60, the resultant sterilized composition may be stored and/or packaged. For example, the composition may be stored frozen, such as at −80° C. or some other suitable temperature, or the composition may be lyophilized and stored under other temperatures, including room temperature or higher temperature.

Once made, a composition as described herein may serve as a tissue graft, wound dressing, cell culture substrate, or other suitable biological structure. Such compositions may be applied to a human or animal patient in any suitable manner, such as by external suturing or gluing, internal implantation, or the like. Compositions as described herein may provide enhanced biocompatibility and healing of any type of damaged or diseased human or animal tissue. The incorporation of bioactive compounds into such compositions may be tailored to promote or inhibit certain biological responses for improved wound healing, repair, and tissue regeneration. Such compositions may thus promote quicker healing through increased cell infiltration and proliferation and decreased inflammation and infection.

The features and advantages are more fully shown by the following examples, which are provided for purposes of illustration, and are not to be construed as limiting the invention in any way. As demonstrated by the examples herein, exposure of amniotic membrane tissue to brief periods of exposure with supercritical carbon dioxide and in the presence of small amounts of peracetic acid may achieve a sterility level of assurance of 10⁻⁶ (that is, a 10⁶ reduction in bacterial load). In addition, the examples herein demonstrate that exposure of amniotic membrane tissue to supercritical carbon dioxide in combination with peracetic acid sterilization treatment may be used to remove bacterial spores—a class of species which is typically difficult to remove. Notably, sterilization may be achieved without significant alteration of tissue architecture, the amounts of pertinent extracellular matrix proteins (type IV collagen, glycosaminoglycans, elastin) present in the tissue, or the biophysical properties of the tissue. Amniotic membrane tissues treated with supercritical carbon dioxide were also found to be excellent substrates for adipose-derived stem cell (ASC) attachment, proliferation and differentiation in vitro. For example, human ASCs attached to various treatment groups after 24 hours of culture continued to proliferate over the next few days and expressed epithelial markers upon differentiation. A wound dressing incorporating supercritical carbon dioxide-treated amniotic membrane materials was also shown to function in vivo as an effective substrate to accelerate wound closure and promote re-epithelialization and vascularization of full thickness wounds. The results indicate that supercritical carbon dioxide exposure can be used to sterilize amniotic membrane tissue grafts while simultaneously preserving the biological attributes which make it appealing for use in numerous clinical and tissue engineering applications.

### Example 1

### Validation of Tissue Sterility

In this Example 1, various conditions were evaluated for efficacy in sterilization of a prevalent bacterial species found on amniotic material and a spore typically resistant to other antimicrobial/sterilization treatments. In preparation of amniotic membrane tissue, placentas from consenting donors undergoing elective caesarean sections were acquired. All donors were negative for infectious diseases, including, but not limited to, human immunodeficiency virus (HIV), hepatitis B and C, and syphilis. To isolate the amniotic membrane, placentas were rinsed in saline and the amniotic membrane was separated from the chorion using blunt dissection under sterile conditions. The amniotic membrane was thoroughly rinsed in saline to remove any remaining chorion, blood clots, and general debris, cut to provide tissue samples of about 2.54 cm², and then placed with the epithelial side up on sheets of 0.45 μm nitrocellulose paper available from Whatman Inc. (Piscataway, N.J.). The amniotic membranes (attached to the nitrocellulose) were sealed in Tyvek™ packaging and kept frozen at −80° C. before further treatment. A similar procedure for obtaining, selecting, and preparing tissues for supercritical fluid treatment was also followed in each of the Examples 2-6 described below. However, in those examples, which involve analysis of structural and/or biochemical comparison of sterilized and native tissues, the tissues were not inoculated with any infectious agent.

In this Example 1, tissue samples were inoculated with 100 μl portions of either bacteria or spore forming bacteria in concentrations to provide about 106 colony forming units. Specifically, the bacterial strains in this example included a clinical isolate of Staphylococcus epidermidis (obtained from San Antonio Military Medical Center, Fort Sam Houston, Tex.) and a spore suspension of Clostridium sporogenes, ATCC strain number 19404, from SGM Biotech/Mesa Labs (Bozeman, Mont.). Because spore-forming bacteria like Staphylococcus epidermidis typically show resistance to traditional sterilization processes such as ethylene oxide, gamma irradiation, and heat/stem sterilization, inactivation of Staphylococcus epidermidis (as shown below in Table 1) may be used to meet industrial sterilization requirements. Staphylococcus epidermidis was cultured in nutrient broth at 37° C. under standard culture conditions, whereas the Clostridium sporogenes suspension was kept at 4° C. prior to tissue inoculation.

The inoculated tissue samples were double-packaged in Tyvek™ packaging and sealed before exposure to supercritical carbon dioxide. In this example, amniotic membrane tissues were treated to supercritical carbon dioxide for a range of times and some of the samples were treated with various amounts of peracetic acid. For each of the inoculants (Staphylococcus epidermidis or Clostridium sporogenes), three replicate samples of amniotic tissue samples were prepared and exposed to the following conditions,

Condition A—10 minutes exposure and 0 ml peracetic acid

Condition B—20 minutes exposure and 0 ml peracetic acid

Condition C—30 minutes exposure and 0 ml peracetic acid

Condition D—10 minutes exposure and 0.5 ml peracetic acid

Condition E—20 minutes exposure and 0.5 ml peracetic acid

Condition F—30 minutes exposure and 0.5 ml peracetic acid

Condition G—10 minutes exposure and 1 ml peracetic acid

Condition H—20 minutes exposure and 1 ml peracetic acid

Condition I—30 minutes exposure and 1 ml peracetic acid

Condition J—10 minutes exposure and 2 ml peracetic acid

Condition K— 20 minutes exposure and 2 ml peracetic acid

Condition L—30 minutes exposure and 2 ml peracetic acid

In addition, untreated amniotic tissue samples, i.e., samples not exposed to supercritical fluid or peracetic acid, were inoculated with either bacteria or spores, and the nutrient broth solutions alone were used as controls.

Exposure of amniotic membrane tissue to supercritical carbon dioxide was performed using a Nova 2200 supercritical fluid sterilizer (NovaSterilis, Lansing, N.Y.). The sterilization unit was custom-made from stainless steel and was designed such that the temperature, pressure, and duration of exposure to supercritical fluid could be controlled with computer software. The processing and sterilization of the amniotic membrane tissue was carried out in an about 20-liter pressure chamber, which houses wire baskets for holding packaged samples as well as an additive pad to which peracetic acid (Sigma, 39% vol/vol peractic acid in acetic acid) was added. As described previously, supercritical fluid may be introduced within the chamber, flow through the pad, solvate peracetic acid (if present), and carry the acid to the tissue composition for sterilization. For all samples in this Example 1, the pressure and temperature of supercritical fluid carbon dioxide was held constant at about 9900 k:Pa (about 1435 psi) and about 35° C., respectively.

After supercritical fluid treatment, amniotic membrane tissue samples inoculated with Staphylococcus epidermidis were placed in tubes containing nutrient broth and incubated at 35° C. under standard culture conditions for 14 days. Similarly, tissue samples inoculated with Clostridium sporogenes were placed in reinforced clostridium broth immediately following sterilization and cultured under anaerobic conditions; that is, in an anaerobic chamber (Whitley MG500; Don Whitley Scientific; Frederick, Md.) under 10% hydrogen/10% carbon dioxide/SO % nitrogen for 14 days. The broth was monitored for bacterial growth by observing turbidity. Furthermore, on days 3, 7, and 14, aliquots (10 μl) of broth from each sample were cultured on either nutrient or reinforced clostridial agar plates for verification of growth (or absence of growth) of Staphylococcus epidermidis or Clostridium sporogenes, respectively.

Amniotic membrane tissue samples may be characterized to be unsterile and positive for growth of bacteria or spore if either (1) turbid culture medium was present or (2) colony forming units grew out on agar plates at any point during the 2-week period of culture incubation. An indication of positive bacterial growth may indicate that the treatment regimen failed to meet industrial sterility standards. The results for the various conditions tested in Example 1 are summarized in Table 1.

TABLE 1

| | | Duration | | |
|---|---|---|---|---|
| | Amount of | Exposure to | | |
| | Peracetic Acid | supercritical | Staphylococcus | Clostridium |
| Condition | (ml) | CO₂ (min) | epidermidis | sporogenes |
| A | 0 | 10 | Fail | Fail |
| B | 0 | 20 | Fail | Fail |
| C | 0 | 30 | Fail | Fail |
| D | 0.5 | 10 | Pass | Fail |
| E | 0.5 | 20 | Pass | Fail |
| F | 0.5 | 30 | Pass | Fail |
| G | 1.0 | 10 | Pass | Fail |
| H | 1.0 | 20 | Pass | Pass |
| I | 1.0 | 30 | Pass | Pass |
| J | 2.0 | 10 | Pass | Pass |
| K | 2.0 | 20 | Pass | Pass |
| L | 2.0 | 30 | Pass | Pass |

In Table 1, an indication of “Pass” denotes the absence of bacterial growth on the agar plates after the prescribed culture time as well as the absence of turbidity in the culture medium. The results show that treatment with supercritical carbon dioxide, when used with suitable amounts of peracetic acid, is an effective method for the sterilization of amniotic membrane tissues and meets industrial sterility standards, specifically a sterility assurance level (SAL) of 10⁻⁶. Moreover, as shown in Table 1, the sterilization of amniotic membrane tissue inoculated with Staphylococcus epidermidis and Clostridium sporogenes can be achieved with short processing time and minimal amounts of peracetic acid sterilizing agent. When using the test conditions described for Example 1, a volume of 0.5 ml of peracetic acid with 10 minutes of exposure to supercritical fluid carbon dioxide was sufficient to inactivate Staphylococcus epidermidis and inhibit contamination over the 2-week culture period; however, an increase in the duration of supercritical fluid carbon dioxide treatment and volume of peracetic acid additive was found to inactivate Clostridium sporogenes. Under the conditions of Example 1, an amount of peracetic acid to inactivate both Staphylococcus epidermidis and Clostridium sporogenes in the shortest time possible (10 min of supercritical fluid carbon dioxide exposure) was found using 2 ml of peracetic acid.

When used in combination with peracetic acid as a sterilizing agent, supercritical carbon dioxide serves to enhance mass transfer of sterilant throughout the amniotic membrane tissue. The combination of peracetic acid and supercritical carbon dioxide acts to sterilize amniotic material to a greater degree than either peracetic acid or supercritical fluid individually. For example, the largest volume of peracetic acid solution in this Example 1 is diluted to a value of 0.01% (2 ml diluted in a 20 liter chamber), a level that is significantly less than other methods that use peracetic acid solutions as a terminal sterilization method. See Wilshaw, S. P., et al., Biocompatibility and potential of acellular human amniotic membrane to support the attachment and proliferation of allogeneic cells, Tissue Eng Part A 14, 463, 2008. Furthermore, combining peracetic acid with supercritical fluid carbon dioxide results in a much shorter exposure time to peracetic acid in order to achieve sterilization of the tissue than using peracetic acid alone, which generally requires anywhere from 3 to 5 hours for sterilization. See von Versen-Hoynck, F., et al., The influence of different preservation and sterilization steps on the histological properties of amnion allografts light and scanning electron microscopic studies. Cell Tissue Bank 5, 45, 2004; Wilshaw, S. P., et al., Biocompatibility and potential of acellular human amniotic membrane to support the attachment and proliferation of allogeneic cells, Tissue Eng Part A 14, 463, 2008; and Rosario, D. J., et al., decellularization and sterilization of porcine urinary bladder matrix for tissue engineering in the lower urinary tract, Regenerative Med 3, 145, 2008.

### Example 2

### Histology and Immunofluorescence Staining

In this Example 2, light microscopy and immunofluorescence staining were used to characterize supercritical fluid treated amniotic membrane tissues. For each technique, samples treated with supercritical carbon dioxide as well as control samples, also referred to as native samples, were collected and analyzed. For those samples subjected to supercritical carbon dioxide, a similar procedure for obtaining, selecting, and preparing tissues for supercritical fluid treatment was used as in Example 1 with the exception that the samples in Example 2 were not inoculated with an infectious agent.

Sterilized samples in Example 2 were treated with supercritical carbon dioxide for time periods of about 10 minutes, about 30 minutes, or about 60 minutes as described below. In addition, for those samples treated with supercritical carbon dioxide, about 2 ml of peracetic acid (˜39% vol/vol peractic acid in acetic acid) was added to an additive pad placed within the sterilization unit unless otherwise noted. The pressure and temperature of the supercritical carbon dioxide treatments were held constant at about 9900 kPa and about 35° C., respectively.

To prepare samples for light microscopy, a portion of tissue was removed from the nitrocellulose paper and rehydrated in saline for 10 min. Small pieces of supercritical carbon dioxide-treated amniotic membrane tissue and a comparison sample of native amniotic membrane tissue were fixed in 10% neutral buffered formalin overnight. The pieces were then embedded in paraffin by standard techniques and sectioned with a microtome. Cut sections (10 μm thick) were stained with a picrosirius red (PSR) stain kit (Polysciences, Inc.; Warrington, Pa.) following standard procedures for polarized light microscopy analyses. Sections were deparaffinized and hydrated with distilled water and stained with hematoxylin. Sections were then incubated in sirius red F3B solution for 1 hour. The stained sections were then washed in 0.01 normal hydrochloric acid (HCl), dehydrated, cleared, and mounted (Histomount, National Diagnostics, Atlanta, Ga.). Images of the stained tissue sections were then acquired with an Olympus BX60 microscope (Center Valley, Pa.) equipped with appropriate filters for polarized light and using a computer software package. A light micrograph of a native amniotic membrane sample using picrosirius red staining is shown in FIG. 5A, and a corresponding micrograph of a supercritical carbon dioxide-treated amniotic membrane sample (10 minute exposure and 2 mls peracetic acid) is shown in FIG. 5B. A light micrograph of a native amniotic membrane sample obtained using polarized light is shown in FIG. 5C, and a corresponding micrograph of a supercritical carbon dioxide-treated amniotic membrane sample is shown in FIG. 5D (10 minute exposure and 2 mls peracetic acid).

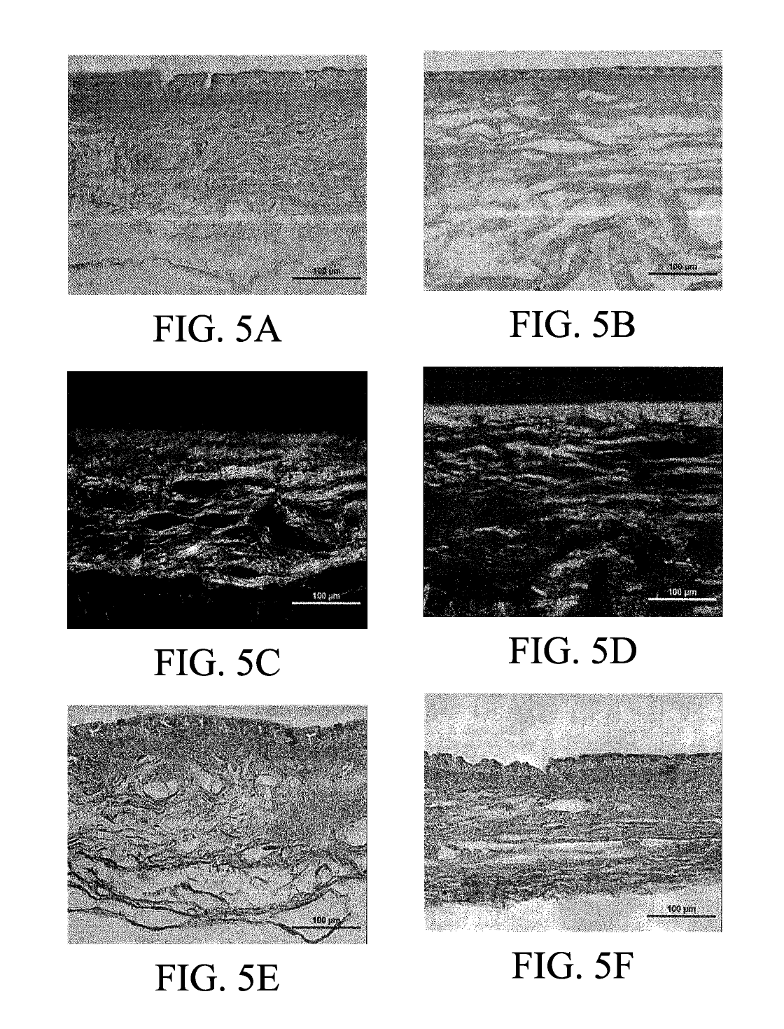

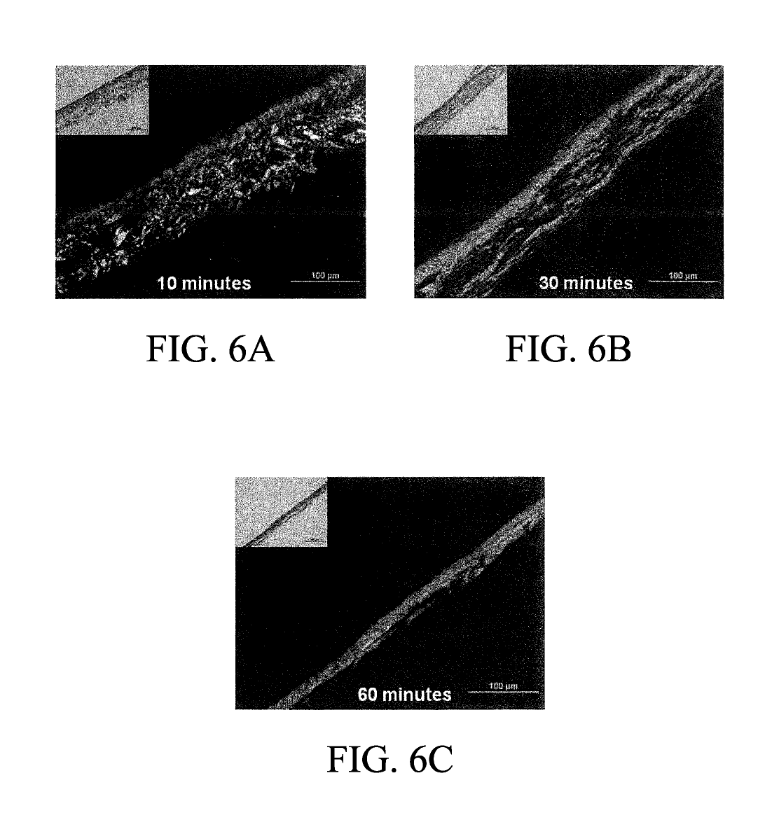

In addition, to further evaluate characteristics of amniotic membrane tissue samples, other samples were exposed to a range of supercritical carbon dioxide treatment times as shown in FIGS. 6A-6C. FIGS. 6A-6C are light micrograph images of samples using picrosirius red staining obtained using polarized light and supercritical carbon dioxide treatment duration periods of 10 minutes (FIG. 6A), 30 minutes (FIG. 6B), and 60 minutes (FIG. 6C). As evident from the micrographs, the sample exposed to a 30-minute exposure period showed only minor changes in structure when compared to the 10-minute exposure. However, the sample exposed to 60 minutes exhibited some loss of structure in the collagenous layers of the membrane tissue.

To prepare samples for immunofluorescence staining, cleared and dehydrated amniotic membrane tissues were rinsed in a solution of tris-buffered saline (TBS, Fisher, Fair Lawn, N.J.) with 0.025% Triton X-100 (Sigma Aldrich, St. Louis, Mo.) and then blocked (to prevent nonspecific binding of the primary antibody to be added) for 2 hours at room temperature using a solution of 10% horse serum (Gibco. Grand Island, N.Y.) and 1% bovine serum albumin (BSA; Sigma Aldrich, St. Louis, Mo.) in TBS. Following blocking, tissue sections were incubated with the primary antibody, a mouse monoclonal antibody specific to type I collagen (Abeam, Cambridge, Mass.), diluted 1:400 in TBS with 1% BSA at 4° C. overnight. Following the primary antibody incubation, sections were rinsed in TBS/0.025% Triton X-100 and then incubated in 0.3% hydrogen peroxide (Henry Schein, Melville, N.Y.) at room temperature for 15 minutes to block endogenous peroxidase activity. Sections were washed in TBS and incubated with a biotinylated horse anti-mouse IgG secondary antibody (1:250 dilution; Vector, Burlingame, Calif.) at room temperature for 1 hour, rinsed in TBS again, then incubated in Vecstatin ABC reagent (Vector, Burlingame, Calif.) at 37° C. for 30 minutes before development with diaminobenzidine (DAB; Vector, Burlingame, Calif.). The sections were finally rinsed in running tap water and counterstained with methyl green (Vector, Burlingame, Calif.) for the visualization of cell nuclei. The stained sections were then dehydrated, cleared, and mounted (Histomount, National Diagnostics, Atlanta, Ga.). Images of the stained tissue sections were acquired with an Olympus BX60 microscope (Center Valley, Pa.) using a computer software package. Light micrographs of samples including immunohistochemical staining for type I collagen are shown in FIG. 5E (native amniotic membrane sample) and FIG. 5F (supercritical carbon dioxide-treated amniotic membrane sample—10 minute exposure and 2 mls peracetic acid).

A comparison between the native and supercritical carbon dioxide-treated amniotic membrane tissue samples of FIGS. 5A-5F indicates that the gross appearance and general structural properties of the tissue extracellular matrix are similar. Specifically, the PSR stain in conjunction with polarized light microscopy was used to enhance the inherent birefringence of collagen molecules, thus allowing for the evaluation of collagen fiber organization of supercritical fluid carbon dioxide-treated tissue as compared to native tissue. Polarization colors observed with PSR staining correspond to collagen fiber thickness and packing density. Thicker, tightly packed collagen fibers exhibit a more intense birefringence of orange to red color, whereas thinner, more loosely packed fibers appear green to yellow. See Junqueira, L. C., Bignolas, G., and Brentani, R. R, Picrosirius staining plus polarization microscopy, a specific method for collagen detection in tissue sections, Histochem J 11, 447, 1979. Notably, the sterilization processing parameters used in this Example 2 did not have a significant effect on the structure and collagen organization of the tissue as indicated by PSR staining (FIGS. 5A-5D). PSR staining of supercritical carbon dioxide-treated amniotic membrane tissue using polarized light revealed dispersed birefringence with green, yellow, and orange/red coloring present throughout most of the tissue regions (FIGS. 5C and 5D). The color and intensity of the birefringence was similar to that of native amniotic membrane. Therefore, the structure of amniotic membrane tissue is substantially maintained after sterilization with supercritical carbon dioxide. In addition, immunohistochemical observation of supercritical carbon dioxide-treated amniotic membrane tissue (FIG. 5F) showed positive staining for the major extracellular matrix protein collagen I throughout the entire cross section of tissue, similar to that of native amniotic membrane tissue (FIG. 5E).

### Example 3

### Fluorescence Microscopy and Scanning Electron Microscopy

In this Example 3, the structure and ultrastructure of amniotic tissue components, including, for example, the amniotic membrane epithelium, was evaluated pre- and post-treatment with supercritical fluid carbon dioxide using both fluorescent microscopy and scanning electron microscopy. For each technique, samples treated with supercritical carbon dioxide (prepared for sterilization as described previously) as well as control samples were collected and analyzed.

The supercritical carbon dioxide-treated samples in Example 3 were exposed for a duration of about 10 minutes. In addition, about 2 ml of peracetic acid (˜39% vol/vol peractic acid in acetic acid) was added to an additive pad placed within the sterilization unit unless where otherwise noted. The pressure and temperature of supercritical carbon dioxide were held constant at about 9900 kPa and about 35° C., respectively.

To prepare samples for fluorescence microscopy, pieces of amniotic membrane tissue were fixed in acetone at −20° C. for 3 minutes, rinsed (3×5 min) in Hank's balance salt solution (HBSS; Life Technologies, Carlsbad Calif.) and then incubated at 37° C. in 10 μg/ml of CellMask™ Deep red plasma membrane stain (Molecular Probes, Eugene, Oreg.) solution for 5 minutes. The labeled amniotic membrane tissues were again rinsed in HBSS before being mounted in prolong gold antifade reagent (Molecular Probes, Eugene, Oreg.) and imaged. Fluorescent images of the cell membranes were obtained using fluorescent microscopy (Olympus BX60 microscope, Center Valley, Pa.) using the appropriate filters (649/666 nm excitation/emission).

To prepare samples for scanning electron microscopy (SEM), control samples and amniotic membrane tissue samples treated with supercritical fluid carbon dioxide were processed using standard procedures. Standard procedures for preparation of samples for SEM analysis are more fully described in Araujo et al. See Araujo, J. C., et al., Comparison of hexamethyldisilazane and critical point drying treatments for SEM analysis of anaerobic biofilms and granular sludge, J Electron Microscopy (Tokyo) 52, '429, 2003. The tissues were fixed in 2.5% phosphate-buffered gluteraldehyde at 4° C. for 1 h, dehydrated in a series of graded alcohols (50%, 70%, 80%, 90%, 95%, and 100%) and dried using hexamethyldisilazane (HMDS, Electron Microscopy Sciences, Hatfield, Pa.). The samples were then sputter-coated with a thin layer (10 nm) of gold and palladium (Anatech; Union City, Calif.) and examined on a Zeiss Sigma VP 40 (Zeiss-Leica, Thornwood, N.Y.) scanning electron microscope. Images of representative areas of the epithelial surface of the AM tissues were acquired.

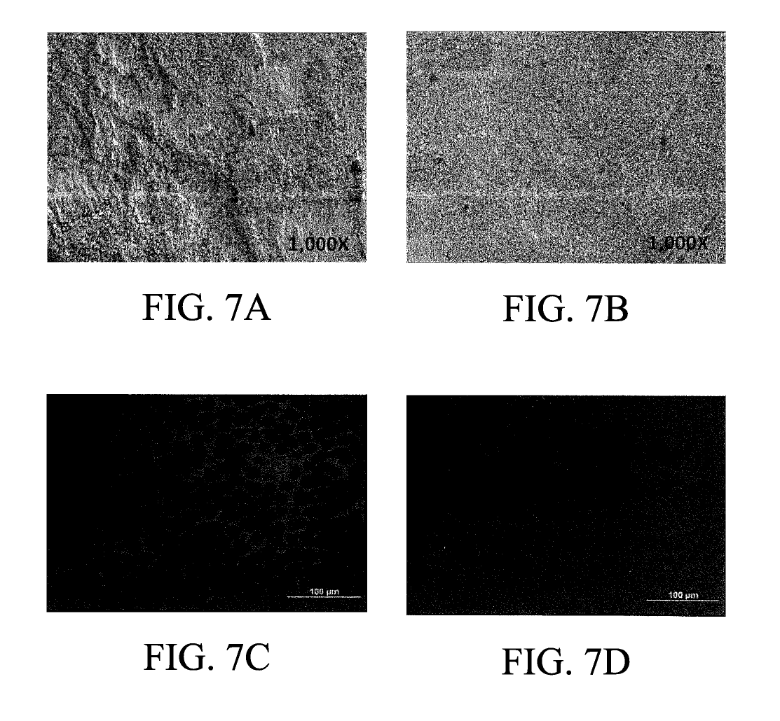

FIGS. 7A and 7B show representative SEM images of the epithelium of native (FIG. 7A) and supercritical carbon dioxide-treated amniotic membrane tissue (FIG. 7B). The epithelium after supercritical fluid carbon dioxide treatment appeared flattened with less distinguishable cellular boundaries as compared to native epithelium, indicating that the sterilization conditions used in this•study affected the epithelial ultrastructure. Because supercritical fluid carbon dioxide treatment has been shown previously to remove lipids from tissues, a plasma membrane stain was used to further characterize the structure of the amniotic epithelium as it pertains to the integrity of the phospholipid bilayer. FIGS. 7C and 7D show representative fluorescence images of the epithelium of native (FIG. 7C) and supercritical carbon dioxide-treated amniotic membrane tissue (FIG. 7D). Fluorescence microscopy revealed an absence of positive staining.

### Example 4

### Calorimetry and FTIR Spectroscopy

In this Example 4, amniotic membrane samples were evaluated pre- and post-treatment with supercritical carbon dioxide using both differential scanning calorimetry (DSC) and Fourier transform infrared (FTIR) spectroscopy. For each technique, samples treated with supercritical carbon dioxide (using conditions described previously) as well as control samples were collected and analyzed.

The supercritical carbon dioxide-treated samples in Example 4 were exposed for a duration of about 10 minutes or about 30 minutes as noted below. In addition, about 2 ml of peracetic acid (39% vol/vol peractic acid in acetic acid) was added to an additive pad placed within the sterilization unit unless where otherwise noted. The pressure and temperature of supercritical carbon dioxide were held constant at about 9900 kPa and about 35° C., respectively.

The thermal transitions of the supercritical fluid car on dioxide-treated amniotic membrane samples and the control samples were analyzed by DSC using a Perkin Elmer DSC7 (Waltham, Mass.). Control and supercritical carbon dioxide-treated amniotic membrane tissues were lyophilized overnight, weighed (5 mg dry weight per sample), then sealed in aluminum pans before being heated at a rate of 30° C./min over a temperature range of 250° C.-300° C. An empty aluminum pan served as the reference for all samples tested. DSC thermograms (shown in Figure SA) were collected using the accompanying Pyris™ software, and the temperatures at which the thermal transition peaks occurred were identified. The transition temperatures, an indicator of the resistance of a material to heat denaturation, were defined as the peak maximum of the resultant endothermic peaks. The results from three separate trials were averaged. A second thermal run on the native tissue was performed to compare the thermal transition of native and denatured tissue samples.

To further assess any changes in the chemical structure of amniotic membranes after treatment with supercritical carbon dioxide, samples were evaluated by means of FTIR spectroscopy. Spectra for native amniotic membranes as well as membranes exposed to and 30 min. of supercritical fluid carbon dioxide were acquired using a Tensor 27 spectrometer (Bruker, Billerica, Mass.). Spectral scanning in the range of 4500-400 cm⁻¹ with a resolution of 4 cm⁻¹ was performed and the absorbance at each wavelength recorded for all of the samples using OPUS™ software. Infrared spectra of both native amniotic membrane (indicated by the solid line) and amniotic membrane subjected to 10 minutes exposure to supercritical carbon dioxide (indicated by a dashed line) are shown in FIG. 8B.

DSC was used to analyze the thermal transitions of native and supercritical fluid carbon dioxide-treated amniotic membrane tissues. Typical thermograms of native and supercritical carbon dioxide-treated amniotic membrane tissues (10 minute exposure and 2 mls. peracetic acid) are shown in FIG. 8A. As the amniotic membrane tissue was subjected to a constant heating rate (30° C./min), the collagen molecules present in the tissue underwent thermal dehydration and structural changes as evidenced by the endothermic peaks that appeared on the DSC curves. The first thermal transition occurred at 114.4±0.68° C. and 113.4±0.88° C. for native and supercritical fluid carbon dioxide-treated membranes, respectively, and was due to a loss of bound water following denaturation of the collagen molecules present in the amniotic membrane. The second thermal transition occurred at 218.4±0.92° C. and 220.7±1.783° C. for native and supercritical fluid carbon dioxide-treated membranes, respectively, as a result of tissue melting and structural decomposition of the amniotic membrane. Moreover, the second thermal run of the denatured native amniotic membrane did not show any characteristic transition peaks, confirming that the amniotic membrane conserved its molecular integrity even after supercritical fluid carbon dioxide and peracetic acid treatment. The DSC data, particularly when viewed together with FTIR data and the collagen degradation analysis discussed further herein, provides evidence that supercritical carbon dioxide treatment with peracetic acid did not cause any major protein degradation within the amniotic membrane tissue matrix.

The molecular organization of the collagen network in the amniotic membrane tissue exposed to supercritical fluid carbon dioxide was also characterized using FTIR spectroscopy. Infrared spectra of native and amniotic membrane tissue exposed to supercritical fluid carbon dioxide are shown in FIG. 8B. Supercritical fluid carbon dioxide treated membranes exhibited amide absorption bands at 1655⁻¹ (amide I), 1554 cm⁻¹ (amide II), and cm⁻¹ (amide III), which are characteristic peaks of collagenous tissue, similar to those observed in the native membrane. Other peaks present in the spectra of both native and supercritical fluid carbon dioxide-treated amniotic membranes include 3315 cm⁻¹. and 2925 cm•1 corresponding to N—H (amine) and C—H (alkane) stretching vibrations (which are associated with lipid alkyl chains), respectively. There were no qualitative differences in the spectral peak positions or the pattern of peaks present for the native and supercritical fluid carbon dioxide treated tissue, thus indicating that the supercritical fluid carbon dioxide treatment does not alter the chemical composition or functional groups of biological molecules present in AM tissue. The ratios of the peak intensities of the amide I, II, and III peaks for native and supercritical fluid carbon dioxide-treated amniotic membranes were calculated to verify this finding.

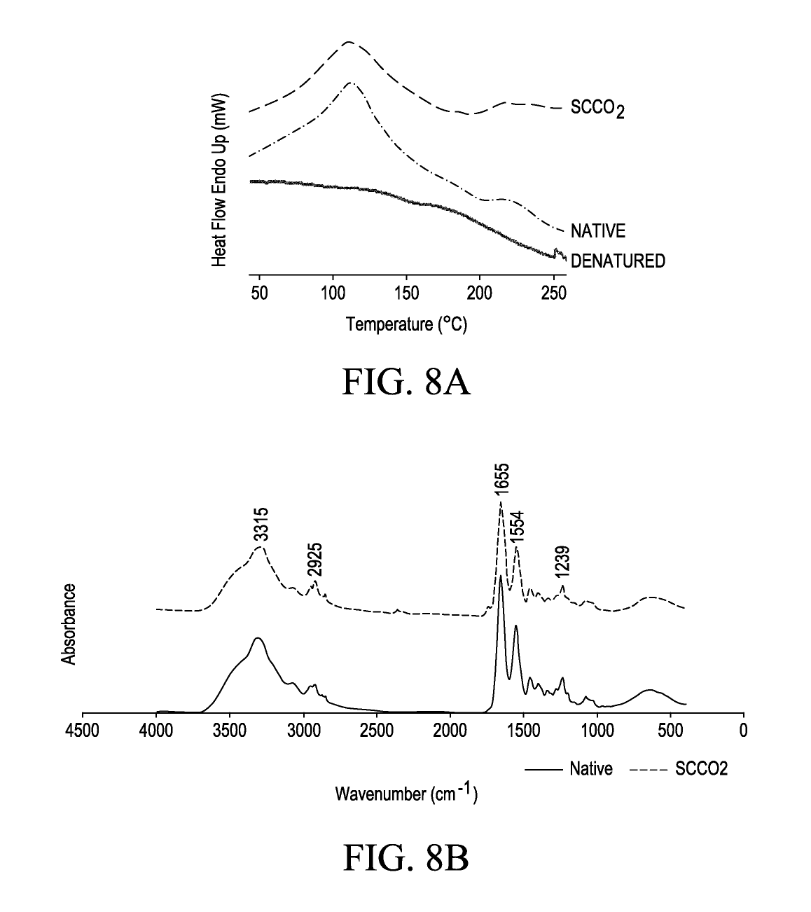

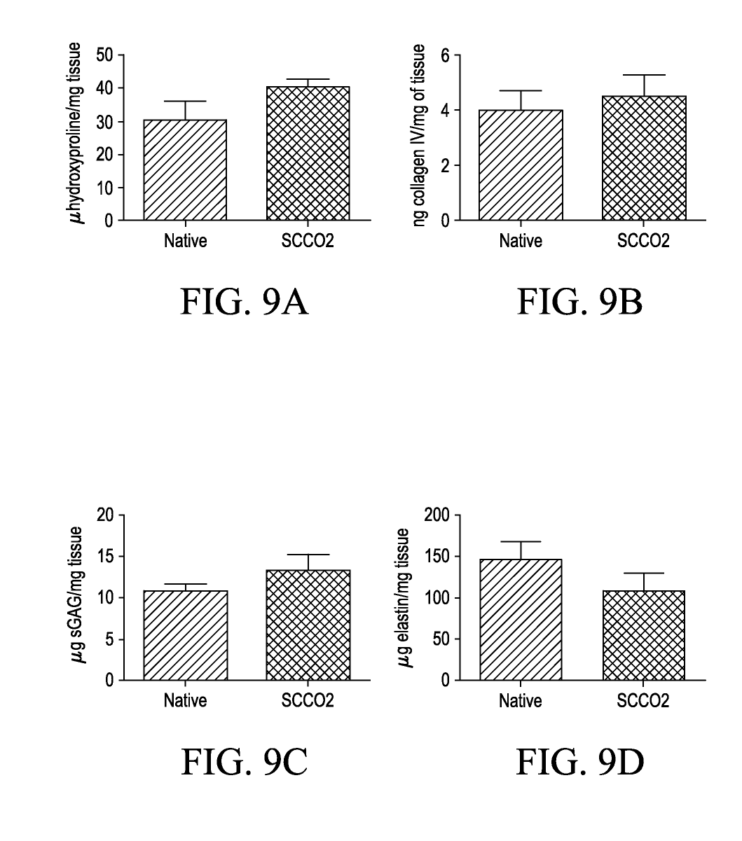

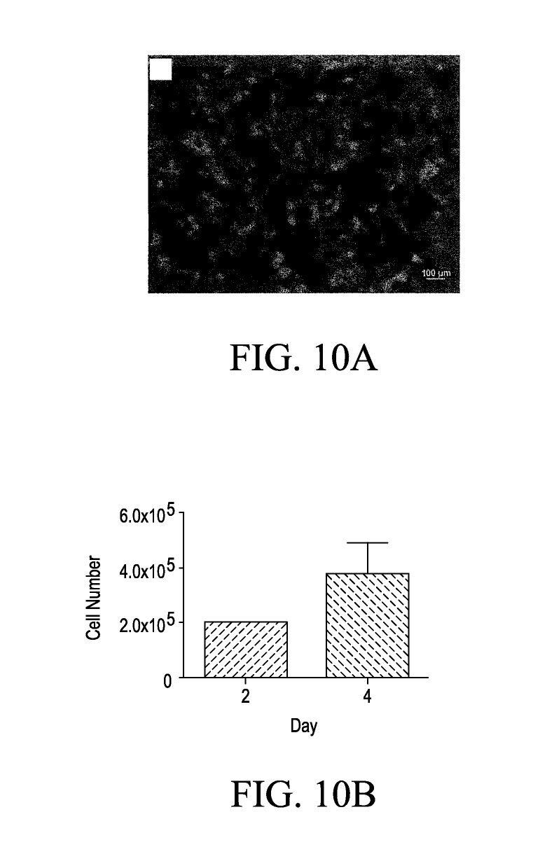

IR spectroscopy is a useful tool for obtaining molecular-level information pertinent to functional groups, chemical bonds, and molecular confirmations of proteins present in normal, physiological, and pathological tissues. FTIR spectra of amniotic membrane tissue treated with supercritical carbon dioxide and peracetic acid did not show any considerable differences in the absorption frequencies and peak intensities in comparison to native tissues. Moreover, FTIR analysis did not show any major change in the spectral frequencies corresponding to amide absorption peaks (amide I, II, and III) typical of amniotic membranes and other collagenous tissues. This information provides evidence that supercritical carbon dioxide/peracetic acid treatment did not cause any major protein degradation within the amniotic membrane matrix. These findings are further validated through DSC analyses (described above. and shown in Fig. SA) and also quantified using a collagen degradation assay (see Example 5 and FIG. 9A-9D). Thermal denaturation involves the breaking of intramolecular bonds of the collagen molecules present in the amniotic membrane tissue, reducing the organized triple helices to a random, amorphous coil form. Degradation to the collagen molecule brought about as a result of processing techniques would result in a decrease in the thermal transition temperatures of the amniotic membranes; however, because no significant changes were observed in the DSC curves between the native and the supercritical carbon dioxide-treated amniotic membrane tissues, it may be concluded that the sterilization treatment is not adversely affecting the hydrothermal stability of the amniotic membrane tissue.

### Example 5

### Biochemical Characterizations

In this Example 5, amniotic membrane samples were evaluated pre- and post-treatment with supercritical fluid carbon dioxide using biochemical assays. For each assay, samples treated with supercritical carbon dioxide (using conditions described previously) as well as control samples were collected and analyzed.

Samples in Example 5 were treated with supercritical carbon dioxide for time periods of about 10 minutes or about 30 minutes as described below. In addition, for each sample about 2 ml of peracetic acid (−39% vol/vol peractic acid in acetic acid) was added to an additive pad placed within the sterilization unit unless otherwise noted. The pressure and temperature of the supercritical carbon dioxide treatments were held constant at about 9900 kPa and about 35° C., respectively.

The amounts of hydroxyproline, type IV collagen, elastin, and glycosaminoglycans (GAGs) present in native (n=6) and supercritical carbon dioxide-treated tissues (n=6) were quantified with a commercially available hydroxyproline assay (BioVision, Mountain View, Calif.), type IV collagen enzyme-linked immunosorbent assay (ELISA) (Exocell, Philadelphia, Pa.), Fastin™ elastin assay (Biocolor, Northern Ireland,” UK), and Blyscan™ sulfated GAG assay (Biocolor, Northern Ireland, UK), respectively, following the manufacturer's recommended procedures. Briefly, tissue samples were lyophilized overnight, and the dry weight was measured. For the hydroxyproline assay, the amniotic membrane tissue was completely solubilized in 12N HCl at 100° C. for 3 h. For the extraction of type IV collagen and the GAGs, the tissue was digested in a papain extraction reagent consisting of 0.2 M sodium phosphate buffer, sodium acetate, ethylenediaminetetraacetic (EDTA) acid, cysteine HCl, and papain at 65° C. overnight. Elastin was extracted by incubating tissue samples in 0.25 M oxalic acid at 60° C. for 1 h. The elastin extraction process was repeated with fresh oxalic acid, and the two extractions were pooled for analysis. The concentrations of hydroxyproline, type IV collagen, elastin, and GAGs contained in each sample tested were determined using a standard curve of light absorbance (560 nm, 450 nm, 513 nm, and 656 nm for hydroxyproline, type IV collagen, elastin, and GAGs, respectively) versus known concentrations of each protein run in parallel with the experimental samples. The data are expressed per milligram of tissue.

The degree of collagen denaturation after supercritical carbon dioxide treatment was also assessed using an a-chymotrypsin assay following previously published procedures. Bank, R. A., et al., A simplified measurement of degraded collagen in tissues: application in healthy, fibrillated and osteoarthritic cartilage, Matrix Bioi 16, 233, 1997. Briefly, lyophilized AM tissue was incubated in 0.1 M tris-HCl containing 1 mg/ml a-chymotrypsin (Sigma Aldrich, St. Louis, Mo.), 1 mM iodoacetamide, and 1 mM ethylenediaminetetraacetic acid (Sigma Aldrich, St. Louis, Mo.) overnight at 37° C. to digest denatured collagen within the matrix. The supernatant, containing the degraded collagen, was solubilized (12 N HCl at 100° C. for 3 h), and the amount of hydroxyproline was determined as outlined above. The amount of hydroxyproline obtained from denatured collagen was expressed as a percentage of the total hydroxyproline content.

The amounts of major ECM components total collagen (represented in terms of hydroxyproline concentration), type IV collagen, elastin, and GAGs present in native and supercritical carbon dioxide-treated amniotic membrane tissue were quantified (FIG. 9A-9D). The amount of hydroxyproline present in the amniotic membrane tissue was not significantly different after exposure to supercritical carbon dioxide, with native membrane containing 30.54±5.48 μg/mg of tissue, whereas supercritical carbon dioxide-treated tissue contained 40.31±2.355 μg/mg of tissue. The amounts of type IV collagen (as determined by ELISA), sulfated GAGs, and elastin present in the supercritical carbon dioxide-treated tissues were not significantly different from that of native tissue. The amount of type IV collagen present in native and supercritical carbon dioxide-treated amniotic membrane tissue was found to be 4±0.7 and 4.5±0.8 ng/mg of tissue, respectively. The amount of sulfated GAGs present in native and supercritical carbon dioxide-treated amniotic membrane tissue was found to be 10.91±0.77 and 13.41±1.91 μg/mg of tissue, respectively. The amount of elastin present in native and supercritical carbon dioxide-treated amniotic membrane tissue was found to be 147.0±20.97 and 109.6±20.66 μg/mg of tissue, respectively.

As noted above, the collagen degradation assay supports both the DSC and FTIR data in concluding that collagen degradation was not occurring and that supercritical carbon dioxide treatment with peracetic acid did not cause any major protein degradation within the amniotic membrane tissue matrix. The amount of hydroxyproline from denatured collagen in native amniotic membrane tissue and amniotic membrane tissue that underwent supercritical carbon dioxide sterilization was not significantly different. Native amniotic membrane tissue consisted of 4% hydroxyproline from denatured collagen while supercritical fluid carbon dioxide treated amniotic membrane tissue contained 5.1% hydroxyproline from denatured collagen.

Native amnion consists of three main structural layers underlying the basement membrane: the compact, fibroblast, and spongy layers composed primarily of type I and III collagens with smaller amounts of collagen types IV-VII. See Meinert, M., et al., Proteoglycans and hyaluronan in human fetal membranes, Am J Obstet Gynecol 184, 679, 2001. In addition, amniotic membrane contains significant amounts of the proteoglycans decorin, biglycan, and hyaluronic acid as well as GAGs and elastin molecules. Collectively, these biomolecules provide tensile strength and impart elasticity that are unique to the amniotic membrane. The data shown indicates that the amounts of total collagen, type IV collagen, GAGs, and elastin present in supercritical fluid-treated anmiotic membrane tissues do not significantly differ from those of native amniotic membrane tissues. Testing on samples exposed to a 30 minute period of exposure to supercritical carbon dioxide also showed that the amounts of total collagen, type IV collagen, GAGs, and elastin present in supercritical fluid-treated amniotic membrane tissues did not significantly change.

### Example 6

### In Vitro Characterization Using Human Adipose-Derived Stem Cells

In Example 6, the biocompatibility of amniotic membrane tissue exposed to supercritical fluid carbon dioxide was evaluated in vitro using human adipose-derived stem cells (hASCs) previously isolated from debrided skin. The hASCs were maintained in MesenPRO RS™ basal medium supplemented with MesenPRO RS™ growth supplement, an antibiotic-antimycotic solution (100 U/ml penicillin G, 100 μg/ml streptomycin sulfate, and 0.25 μg/ml amphotericin B), and 2-mM L-glutamine (Gibco, Invitrogen, Carlsbad, Calif.) under standard cell culture conditions (that is, a sterile, 37° C., humidified, 5% CO2/95% air environment). Cells at passage 2-4 were used in the current experiments.

Supercritical carbon dioxide-treated amniotic membrane tissues measuring 2.54 cm in diameter were placed inside 12-well cell culture inserts (BD Biosciences, Franklin Lakes, N.J.) with the epithelial side facing upward. The hASCs fluorescently labeled with carboxyfluorescein diacetate, succinimidyl ester (CFSE; Life Technologies, Eugene, Oreg.) were seeded (50,000 cells per insert) on top of the amniotic membranes and maintained, submerged in MesenPRO culture medium, over a period of 4 days. Cell attachment was assessed 24 hours post seeding, and fluorescent images were taken with an Olympus 1X71 inverted microscope equipped with reflected fluorescence system (Olympus America Inc., Center Valley, Pa.).

Proliferation was monitored over the 4-day period using the colorimetric MTT (3-(4,5-dimethylthiazol-2-yl)-2,5-diphenyltetrazolium bromide) metabolic activity assay (Life Technologies; Eugene, Oreg.). Briefly, on days 1, 2, 3, and 4, 25 μl of MTT (5 mg/ml) was added to each cell culture insert. The hASCs were allowed to reduce the tetrazolium salt to formazan over a period of 4 hours (at 37° C.). At that time, the purple formazan was extracted from the cells and solubilized using 250 111 of dimethyl sulfide (DMSO, Sigma, St. Louis, Mo.). The optical density of the resulting solution was determined by measuring the absorbance at 570 nm with 630 nm as reference using a microplate reader (Synergy MX, BioTek, Winooski, Vt.).

The biocompatibility of amniotic membrane tissue exposed to supercritical fluid carbon dioxide with regards to cell adherence and proliferation was evaluated in vitro using hASCs. The hASCs were fluorescently labeled with CFSE and cultured on SCC02 treated AM for up to 4 consecutive days. Representative fluorescent micrographs show that the hASCs attached to supercritical carbon dioxide-treated amniotic membrane tissue after 24 hours of culture (FIG. 1OA) and continued to proliferate over the next few days (FIG. 1OB) as determined by MTT. By day 4, the hASCs had more than doubled in population, thus indicating that the sterilized amniotic membrane tissue is a good substrate for hASC culture.

### Example 7

### In Vive Characterization of Supercritical Carbon Dioxide-Treated Amniotic Membrane Tissues'

In Example 7, the performance of a wound dressing including amniotic membrane tissue was characterized. The constructs used for the in vivo experiments were prepared prior to surgery and fabricated using either amniotic membrane tissue or amniotic membrane tissue seeded with hASCs. To obtain amniotic membrane tissue for the wound dressing, placental material was obtained from consenting donors (negative for infectious diseases, including, but not limited to, human immunodeficiency virus (HIV), hepatitis B and C, and syphilis) undergoing elective caesarean sections. To isolate the amniotic membrane, placentas were rinsed in saline and the amniotic membrane was separated from the chorion using blunt dissection under sterile conditions. The amniotic membrane was thoroughly rinsed in saline to remove any remaining chorion, blood clots, and general debris, cut to provide tissue samples, and then placed with the epithelial side up on sheets of 0.4 μm nitrocellulose paper available from Whatman Inc. (Piscataway, N.J.). The amniotic membranes (attached to the nitrocellulose) were sealed in Tyvek™ packaging and kept frozen at −80° C. before further treatment. Conditions for exposure of the membranes to supercritical carbon dioxide were identical to the samples previously described in Example 2. Following exposure, amniotic membrane tissue was removed from the nitrocellulose paper, rehydrated in saline for about 10 minutes and cut into circular pieces for further processing.

The cell-seeded amniotic membrane wound dressing (hASCs-AM) was then prepared by first securing 4.5 cm diameter circular pieces of amniotic membrane tissue in cell crowns (6-well plate format; Scaffdex, Finland) with the basement membrane facing downwards, towards the bottom surface of the cell culture plate. The hASCs (100,000 cells/construct) were then added to the inside of the cell crown, seeded on the epithelial surface of the amniotic membrane, and then incubated (37° C., 5% CO2/95% air) in complete MesenPRO culture medium for 18 hours to allow for cell attachment.

To evaluate the wound dressings in vivo, male mu nude athymic rats (175-250 g) were obtained from Harlan Laboratories (Indianapolis, Ind.) and housed in the United States Army Institute of Surgical Research animal care facility. Rats were allowed to access water and chow ad libitum. On the day of surgery, a 1.4 mm diameter full thickness excision wound was created on the dorsum down to the panniculus of the rat. Following the creation of the excision wound, animals received one of three treatments: 250 μl saline, AM wound dressing, or the hASCs-AM wound dressing. All of the wounds were covered with DuoDERM transparent film dressing (3M, St. Paul, Minn.) and observed for up to 16 days.

On days 4, 8, 12 and 16 animals from each treatment group were euthanized in accordance with ethical standards and biopsies of the wound beds, including the healed area of skin around the wound, were harvested and prepared for histological analysis. Briefly, the harvested tissue sections collected at the time of euthanasia were fixed in 10% neutral buffered formalin overnight, dehydrated in a series of ethanol and blocked in paraffin following standard embedding procedures. Cut sections 7 μm thick were stained with Masson's Trichrome stain (MTS), and images of the stained tissue sections were acquired with an Olympus BX60 microscope (Center Valley, Pa.) using the DP Controller™ software package.

The percent wound closure was calculated from wound area assessments acquired on days 4, 8, 12 and 16. This was done by photographing the wound area at the prescribed time points, calculating the wound area in pixels using the Adobe Photoshop software package, and then converting the number of pixels to mm². The percent wound area was. calculated using the following formula,

[(WAO−WAi)/WAO]×100

where WAO and WAi represent the original area of the wound, and the area of the wound at each assessment time point, respectively.

Statistical analyses of numerical results were performed using the GraphPad Prism™ (GraphPad Software; Inc. San Diego, Calif.) statistical software package. Numerical data are expressed as mean±standard error of the mean (mean±SEM). Comparisons between groups were made using at-test with p<0.05 considered statistically significant.

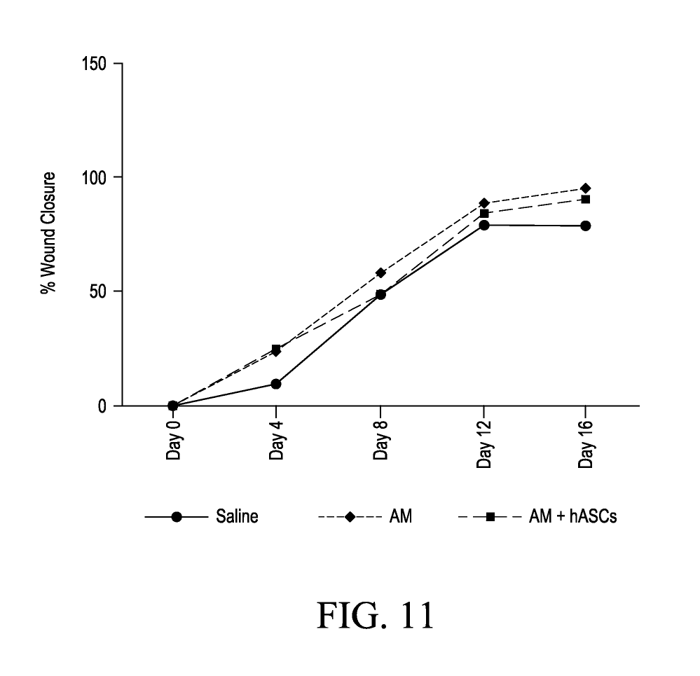

As described herein, rat excision wounds treated with amniotic membrane dressings (with or without hASCs) were found to accelerate wound closure as observed over a period of 16 days. Wound areas obtained on days 4, 8, 12 and 16 were used to calculate the percentage of wound closure of full thickness excision wounds treated with either saline, the AM or hASCs-AM wound dressings (FIG. 11). For all groups, a slower initial phase of healing was observed at day 4, while a period of significantly faster healing occurred through day 12. All of the treatment groups exhibited greater than 90% wound closure (95.29% and 90.4% for the AM or hASCs-AM wound dressing treatment groups, respectively); however, the saline treatment group showed differences in healing, achieving only 78.7% wound closure by the end of the study.

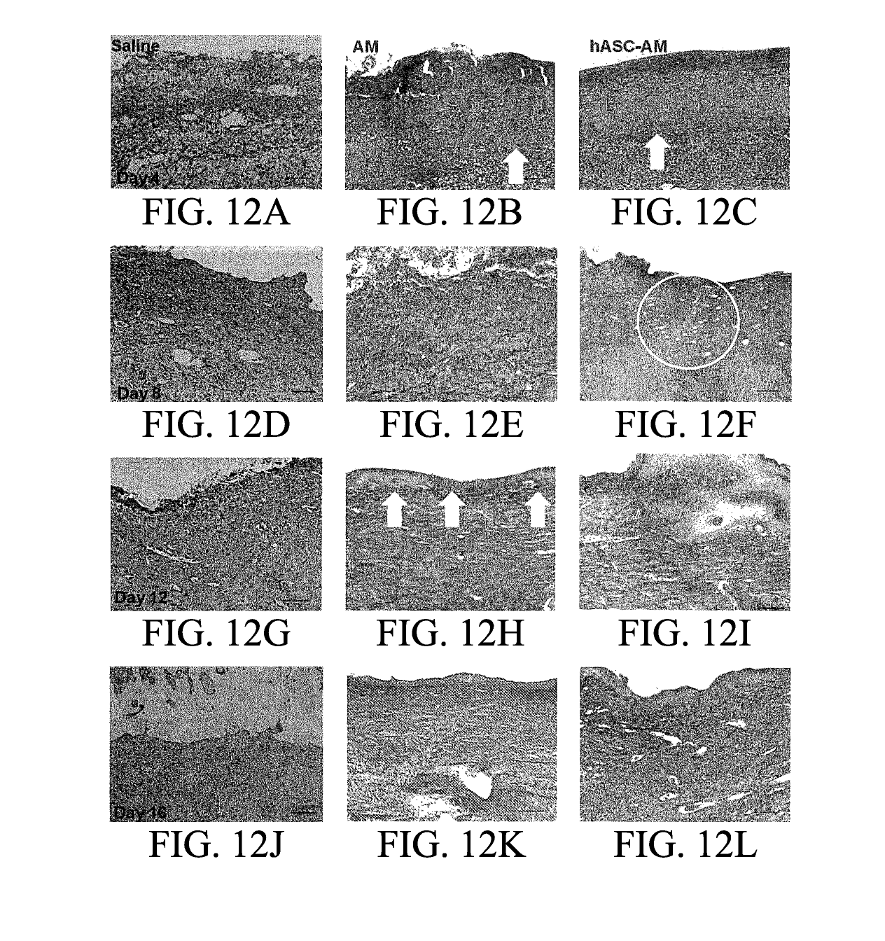

To further investigate the contributions of the hASCs-AM dressings on the wound healing process, a rat full thickness excision wound model was implemented in this study. On days 4, 8, 12 and 16 histological sections of the excised tissues from the wound beds were stained with Masson's Trichrome stain and evaluated microscopically for re-epithelialization, granulation tissue formation and vascularization (FIG. 12). The light micrographs of FIG. 12 show full thickness excision wounds treated with saline, AM wound dressings or hASCs-AM wound dressings monitored over a period of 16 days. The extent of wound healing was evaluated histologically for granulation tissue formation andre-epithelialization at days 4, 8, 12 and 16. Bold arrows in the day 4 panel of images indicate integration of the matrix material into the wound bed. Paraffin sections were stained with MTS. The scale bar in FIG. 12 is 200 Gun.

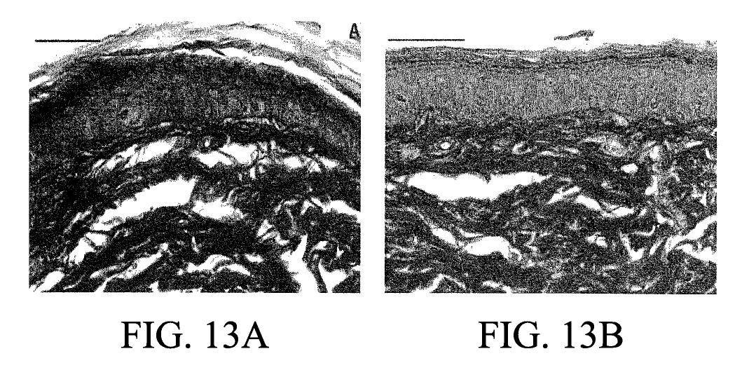

MTS stained sections showed integration of the applied matrices within the wound bed in the treatment groups by day 4 (as indicated by the bold arrows in FIG. 12). By day 8, all of the treatment groups showed granulation tissue formation, as•evidenced by deposition of nascent collagen (blue coloration of the MTS stained sections). However, the treatment groups that involved AM (with or without hASCs) exhibited more mature granulation tissue formation, that is, the organization of the collagen filers formed closely resembled that of native tissue as shown in FIG. 13A-13B. FIG. 13A-13B shows a high magnification light micrograph detailing the epidermis of normal rat skin (FIG. 13A) and the epidermis of a wound treated with the AM wound dressing (without hASCs) after 12 days of healing (FIG. 13B). Stratification of the epidermis in the treatment group (FIG. 13B) is similar to that of normal skin. In addition, the organization of collagen bundles in the treatment group (FIG. 13B) also resembles that of normal rat skin. In FIG. 13A-13B, sections were stained with MTS, scale bar±100 μm. Areas of re-epithelialization were also apparent by day 12 for the AM and the hASCs-AM wound dressing groups. Within the treatment groups, complete re-epithelialization of the wound bed was observed earlier (by day 12) for the AM treatment group as compared to treatment with saline alone. High magnification images of the newly formed epidermis at day 12 revealed close resemblance to that of mature epidermis; more specifically, multiple layers of keratinocytes are easily identifiable (FIG. 12).

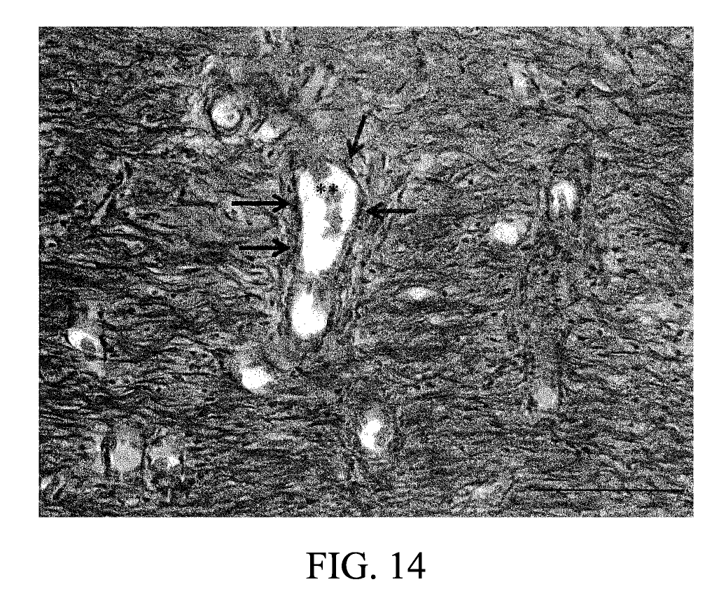

In addition to re-epithelialization, excision wounds treated•with hASCs-AM wound dressings exhibited significant vascularization (FIG. 14). FIG. 14 shows another high magnification light micrograph. It can be clearly seen from this light micrograph of the day 12•tissue section that the vascular structures in the hASCs-AM wound dressing group had matured into functional, patent blood vessels. This is evidenced by the organization of a monolayer of cells forming an outline of the vessel wall (bold black arrows), as well as red blood cells contained within the structures (asterisk).

While many examples in this description refer to compositions and methods thereof, it is understood that those compositions and methods are described in an exemplary manner only and that other compositions and methods may be used. For example, any feature described for one embodiment may be used in any other embodiment. Additionally, other materials and other method steps may be used, depending on the particular needs. Although the foregoing specific details describe certain embodiments, persons of ordinary skill in the art will recognize that various changes may be made in the details of these embodiments without departing from the spirit and scope of this invention as defined in the appended claims and other claims to be drawn to this invention, considering the doctrine of equivalents. Therefore, it should be understood that this invention is not limited to the specific details shown and described herein.

1. A sterilized human tissue preparation for tissue engineering comprising a decellularized portion of a biological tissue comprising amniotic membrane, wherein the amniotic membrane is impregnated with a bioactive agent and has a sterility assurance level of 10−6.

2. The sterilized human tissue preparation of claim 1 wherein the bioactive agent is impregnated within said decellularized portion of the amniotic membrane.

3. The sterilized human tissue preparation of claim 1 wherein said bioactive agent comprises an aminoglycoside antibiotic, glycopeptide antibiotic, or a combination thereof.

4. The sterilized human tissue preparation of claim 1 wherein said bioactive agent comprises gentamicin or vancoymin.

5. The sterilized human tissue preparation of claim 1 wherein the bioactive agent comprises stem cells.

6. A sterilized human tissue preparation useful for tissue engineering comprising a processed human amniotic membrane tissue, wherein the processed amniotic membrane tissue comprises an intact extracellular matrix, and wherein said tissue preparation has a sterility assurance level of 10⁻⁶.

7. The sterilized human tissue preparation of claim 6 further comprising at least one bioactive agent, wherein said at least one bioactive agent is impregnated within said amniotic membrane.

8. The sterilized human tissue preparation of claim 6 wherein the bioactive agent is selected from the group consisting of: human adult stem cells, an aminoglycoside antibiotic, and a glycopeptide antibiotic.

9. The sterilized human tissue preparation of claim 6 wherein the bioactive agent is gentamicin or vancoymin.

10. The sterilized human tissue preparation of claim 6 wherein said extracellular matrix is substantially intact.

11. The sterilized human tissue preparation of claim 6 wherein said amniotic membrane has a sterility assurance level of at least 10⁻⁶.

12. The sterilized human tissue preparation of claim 6 wherein the bioactive agent comprises adult human stem cells.

13. The sterilized human tissue preparation of claim 6 wherein the bioactive agent comprises human adipose derived adult stem cells.

14. The sterilized human tissue preparation of claim 6 wherein said composition is essentially free of Clostridium and Clostridium spores.
